(19) United States
(12) Patent Application Publication
(10) Pub. No.: US 2023/0225315 A1
(43) Pub. Date: Jul. 20, 2023

(54) METHODS OF DELIVERING PLANT VIRUS-BASED NANOPESTICIDES

(71) Applicant: CASE WESTERN RESERVE UNIVERSITY, Cleveland, OH (US)

(72) Inventors: Nicole F. Steinmetz, San Diego, CA (US); Paul L. Chariou, Cleveland, OH (US)

(21) Appl. No.: 17/425,909

(22) PCT Filed: Jan. 27, 2020
(86) PCT No.: PCT/US20/15239

§ 371 Date: Jul. 26, 2021

Related U.S. Application Data
(60) Provisional application No. 62/796,849, filed on Jan. 25, 2019.

(51) Int. Cl. A01N 25/28; A01N 63/40; A01P 5/00

(52) U.S. Cl. CPC A01N 25/28; A01N 63/40; A01P 5/00

(57) ABSTRACT

A method of delivering an agricultural composition to a plant including determining a dose of the agricultural composition required to deliver a treatment effective amount of the at least one agrochemical agent to a target soil depth using a computational model, the agricultural composition including a plurality of plant viral nanoparticles (VNPs) and/or virus-like particles (VLPs) and at least one agrochemical agent, and applying the determined dose of the agricultural composition to the plant.

### RELATED APPLICATION

This application claims priority to U.S. Provisional Application 62/796,849, filed Jan. 25, 2019, the subject matter of which is incorporated herein by reference in its entirety.

### GOVERNMENT FUNDING

This invention was made with government support under Grant No. EB021911 awarded by The National Institutes of Health and DMR1452257, awarded by the National Science Foundation. The United States government has certain rights to the invention.

### TECHNICAL FIELD

Embodiments described herein relate to agricultural compositions and methods of delivering determined doses of the agricultural compositions to plants.

### BACKGROUND

Pesticides are needed to protect our crops and thus maximize crop yields. However, the efficacy of chemical pesticides is limited by their physicochemical properties (instability and strong binding to organic matter in soil), which can render them inactive or prevent their accumulation at the root level, where many pests reside. Large doses are applied to compensate, resulting in the accumulation of pesticide residues in soil, water and agricultural products. Long-term exposure to these chemicals is a risk to human health and threatens the biodiversity of an already fragile ecosystem. Precision farming methods are therefore needed to deliver pesticides in a more controlled manner.

For example, plant parasites are a major burden to the global agricultural industry. Among them, the United States Department of Agriculture (USDA) has highlighted several species of insects and worms (i.e., moths, beetles, fruit flies, grasshoppers, ants, and nematodes) as the most common and devastating parasites. Plant parasites either directly injure crops by feeding on them or indirectly cause injury through the transmission of bacteria, viruses, and fungi.

Endoparasitic plant nematodes feed on the crop roots, causing distinctive root swellings commonly referred to as galls. Gall formation impairs the root conduction of water and growth nutrients into the rest of the plant, resulting in lower crop yields. In addition, galls often promote crack damages in the roots and increase the plant vulnerability to secondary infections.

The root-knot Meloidogyne spp, the potato cyst Globodera spp, and the soybean cyst Heterodera glycines are the most damaging and widely spread plant parasitic nematodes. Combined they can infect more than 3000 plant species, including bananas, corn, cotton, potatoes, lettuce, and tomatoes. While crop nematode infestation is relatively easy to diagnose (e.g., dig up a few plants and examine the roots for gall formation), treatment options are limited.

In most countries, crop rotation is frequently employed to selectively control plant parasitic nematode infestations. Nonetheless, the wide host range of root-knot nematodes limits the choice of alternate crops to a few species, yielding little to no revenue. Genetically modified crops resistant to nematodes are an economically and environmentally viable alternative. Unfortunately, genetic resistance to plant parasitic nematodes is selective to specific nematode species, limited to a few crops, and takes years to engineer.

While these aforementioned control strategies can reduce the burden of plant parasitic nematodes on most crops, their efficacy and economic benefits are no match to the use of nematicides. The first generation of nematicides rely on highly toxic and volatile fumigants, such as methyl bromide, but their use has declined due to environmental (e.g., thinning of the ozone layer and undiscriminating killing of animals such as bees) and health (e.g., reproductive sterility and cancer) concerns. Alternatively, nonfumigant nematicides, such as organophosphates, carbamates, and bionematicides, have been employed. Their efficacy, however, is limited by their ability to diffuse through soil, which is dependent on the amount of organic matter, moisture, and the soil structure (e.g., grain size and soil density). To be effective, nonfumigant nematicides must persist long enough and in concentrations equivalent to the nematode lethal dose at root level. Extended persistence in such doses increases the risk of chemical contamination of crops, soil, and groundwater. Therefore, there is a critical need to resolve soil mobility issues of nematicides to enhance their agrochemical efficacy, reduce their indiscriminate use, and ensure their safe application.

### SUMMARY

Embodiments described herein relate to methods of delivering agricultural compositions to plants. The methods include using a computational model to determine a dose of the agricultural composition required to deliver a treatment effective amount of at least one agrichemical agent to a targeted depth of a plant and applying the determined dose of the agricultural composition to the plant. Agricultural compositions include plant viral nanoparticles (VNPs) and/or virus-like particles (VLPs) thereof and at least one agrochemical agent.

The computational model can be defined by a set of one or more parameters. In some embodiments, the set of parameters can include one or more of the following parameters, a dispersion constant of the plant VNP or VLP through soil, a dispersion constant of the agrochemical agent through soil, a rate constant of VNP or VLP absorption to soil, a rate constant of agrochemical agent absorption to soil, and a rate constant of an agrochemical agent release from the VNP or VLP in fluid.

In some embodiments, the computational model can be used to predict the transport behavior, or soil mobility, of an agrochemical agent associated with plant VNP and/or plant VLP. In some embodiments, the plant VNPs and/or VLPs improve the biodegradability, stability, permeability, soil mobility, and/or dispersion of the at least one agrochemical agent in soil.

In some embodiments, the treatment effective amount is the amount capable of maintaining an IC₅₀ concentration of an agrochemical agent at the target soil depth beneath the surface for at least 24 hours.

Plant VNPs can nonpathogenic or pathogenic plant virus particles. The plant VNPs and/or VLPs can have a variety of geometries. In some embodiments, plant VNPs and/or VLPs include rod-shaped plant VNPs and/or VLPs. In some embodiments, the VNPs and/or VLPs include Virgaviridae virus particles. In other embodiments, the VNPs and/or VLPs can include VNP and/or VLP of the Tobamovirus species. In certain embodiments, the VNPs and/or VLPs can include tobacco mild green mosaic virus (TMGMV) particles and VLPs thereof.

In some embodiments, plant VNPs and/or VLPs include icosahedral shaped plant VNPs and/or VLPs. In some embodiments, the VNPs and/or VLPs can include VNPs and/or VLPs selected from the group consisting of plant Picornavirus and Tymovirus virus particles. In some embodiments, the plant Picornavirus virus particles can include a cowpea mosaic virus (CPMV) or VLP thereof. In some embodiments, the plant Tymovirus virus particles can include a physalis mottle virus (PhMV) or VLP thereof.

The agrochemical agent can be conjugated to an interior and/or exterior surface of the VNPs and/or VLPs. For example, at least one agrochemical agent can be covalently bound to chemically modified amino acid residues on the interior or exterior surface of the plant VNPs and/or VLPs. In some embodiments, the at least one agrochemical agent is conjugated to the exterior surface of the plant VNPs and/or VLPs via a linker, such as a labile ester cleavable linker.

The at least one agrochemical can also be encapsulated within an interior surface of the plant VNPs and/or VLPs. In some embodiments, the at least one agrochemical is encapsulated by mixing plant VNPs and/or VLPs with a molar excess of the at least one agrochemical agent.

In some embodiments, the target soil depth is the depth of the rhizosphere region or the root level of the plant. In some embodiments, the target soil depth is within about 4 cm from the soil surface.

Agricultural compositions can be applied to the plant via spraying, atomizing, dusting, scattering, and/or pouring. Therefore, the agricultural composition can include a sprayable composition. In some embodiments, the agricultural composition further includes a water carrier.

The agrochemical agent of an agricultural composition can be selected from the group consisting of nematicides, fungicides, herbicides, pesticides, acaricides, rodenticides, plant growth regulators, nutrients, pest repellents, and combinations thereof. In some embodiments, the agrochemical agent includes a nematicide, such as abamectin.

In some embodiments, the treatment effective amount of the at least one agrochemical agent is the amount effective to combat nematode parasitism. Nematode parasitism can be caused by a nematode selected from the group consisting of Meloidogyne root knot nematodes, Globodera and Heterodera cyst nematodes; Pratylenchus lesion nematodies, Dietylenchus stem and bulb nematodes, Tylenchulus citrus nematodes, Xiphinema dagger nematodes, Radopholus burrowing nematodes, Rotylenchulus reniformis nematodes, Helicotylenchus spiral nematodes, and Belonolaimus sting nematodes.

Plants to which an agricultural composition is delivered can include monocots or dicot plants. In some embodiments, the plant is selected from the group consisting of wheat, corn (maize), soybean, cotton, cassava, potato, sweet potato, bananas, citrus, strawberries, tomato, coffee, carrots, peppers, turf grass, and greenhouse ornamentals.

### BRIEF DESCRIPTION OF THE DRAWINGS

FIG. 1 illustrates a schematic showing the combined experimental and computational approach to assess nanopesticide transport through soil. The virus-based and synthetic nanoparticles are depicted to scale in the top left corner. Labelled nanoparticles were injected as a bolus at the top of the soil column, and moved through the column at a constant flow rate. At the bottom of the column, particles were collected as 500-μl fractions. The mass of the eluted virus-based nanoparticles was determined by SDS-PAGE and the synthetic nanoparticles were imaged as droplets on Parafilm using the FluorChem R imaging system under MultiFluor red light. Experimental data were imported into MATLAB for comparison with the output of the computational model.

FIGS. 2(A-B) illustrate cargo release from nanoparticles during dialysis. A, Schematic representation of infused-dye release from (left to right) TMGMV, CPMV, PhMV, MSNP and PLGA. The dialysis membrane pores are large enough to allow the free movement of Cy5 but small enough to prevent nanoparticle diffusion. The number of arrows reflects the rate of dye release from each nanoparticle in a semi-quantitative manner B, Corresponding plot of Cy5 cumulative release from each nanoparticle as a function of time. The approximate half-life data (time required to release 50% of the dye) are shown at the bottom right corner of the graph.

FIGS. 3(A-E) illustrate plots showing experimental transport of nanopesticides and pesticides through soil. A, Cumulative mass of bare, Cy5-conjugated, and Cy5-infused nanoparticles exiting the soil column as a function of soil depth. B, Corresponding cumulative moles of conjugated Cy5 and infused Cy5 exiting the soil column. C, Mass distribution of nanoparticles as a function of time at a given soil depth. D, Corresponding mole distribution of Cy5-infused and E, Cy5-conjugated particles as a function of time for a given soil depth.

FIGS. 4(A-C) illustrate plot showing theoretical transport of nanoparticles through soil. A, The empirical output of TMGMV, CPMV, PhMV, MSNP and PLGA is used as a reference. B, Computational modelling of nanoparticle transport through soil. D_{NP} and k_{NPS} were optimized for each depth. C, Corresponding model of nanoparticle transport through soil using the average value of D_{NP} and k_{NPS} obtained in B.

FIGS. 5(A-C) illustrate plots showing theoretical transport of Cy5 through soil. A, The empirical output of Cy5 infused into TMGMV, CPMV, PhMV and MSNP used as a reference. B, Computational modelling of Cy5 transport through soil following nanoparticle infusion. C, Corresponding model output of free Cy5 transport through soil.

FIG. 6 illustrates plots showing theoretical treatment of a crop infected with nematodes using TMGMV-abamectin. Each curve represents the temporal concentration distribution of abamectin conjugated to TMGMV at a soil depth equal to 24 cm as a function of the irrigation flow rate (Q). The corresponding minimal dose of TMGMV (m) that must be applied on the crop to maintain the IC₅₀ of Abamectin is indicated.

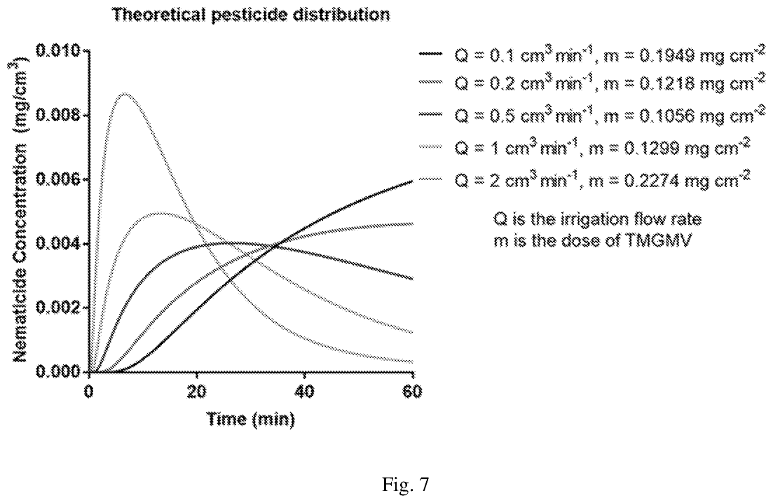

FIGS. 7(A-C) illustrate Cy5 conjugation to TMGMV, PhMV, CPMV and MSNP. The schematics show chemical conjugation of Cy5 to A, the surface-exposed tyrosine residues of TMGMV using diazonium chemistry followed by click chemistry, B, the surface exposed lysine residues of PhMV and CPMV using NHS chemistry, and C, the carboxylate groups of MSNP via EDC and click chemistry.

FIG. 8 illustrates the distribution of free Cy5 in the soil.

FIG. 9 illustrates TEM images and corresponding size distribution of viruses that were leached through the soil column at different soil depths.

FIG. 10 is a schematic of the reaction mechanisms of nanoparticles and pesticides in soil.

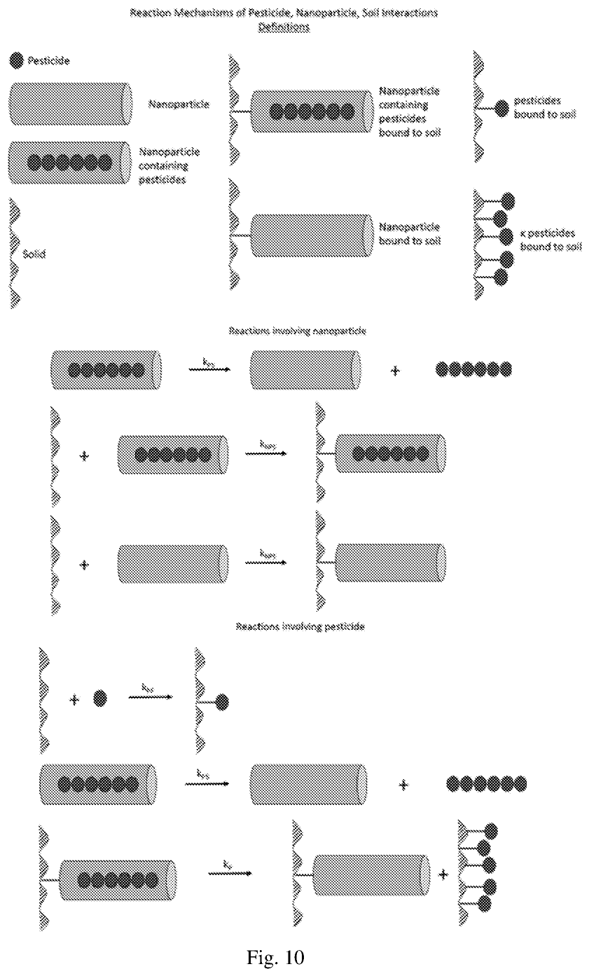

FIG. 11 illustrates free Cy5 model output.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this invention belongs.

The term “about” as used herein when referring to a measurable value such as an amount, a temporal duration, and the like, is meant to encompass variations of +−20% or 110%, more preferably +−5%, even more preferably +−1%, and still more preferably +−0.1% from the specified value, as such variations are appropriate to perform the disclosed methods.

The term “effective amount” refers to an amount of an agent that is sufficient to provide a desired effect. An effective amount in any individual case may be determined by one of ordinary skill in the art using routine experimentation.

The term “nematode” as used herein includes, but is not limited to, plant-parasitic nematodes such as Meloidogyne root knot nematodes, Globodera and Heterodera cyst nematodes, Pratylenchus lesion nematodes, Dietylenchus stem and bulb nematodes, Tylenchulus citrus nematodes, Xiphinema dagger nematodes, Radopholus burrowing nematodes, Rotylenchulus reniformis nematodes, Helicotylenchus spiral nematodes, and Belonolaimus sting nematodes.

The term “plant” as used herein generally refers to vascular plants. “Plant” refers to both whole plants and parts thereof, such as stems, leaves, flowers, fruit, tubers, seeds, roots, etc.

The term “plant propagation material” is understood to denote all the generative parts of the plant, such as seeds, which can be used for the multiplication of the latter and vegetative plant materials, such as cuttings and tubers (for example, potatoes). Accordingly, as used herein, part of a plant includes propagation material. There may be mentioned, e.g., the seeds (in the strict sense), roots, fruits, tubers, bulbs, rhizomes, parts of plants. Germinated plants and young plants, which are to be transplanted after germination or after emergence from the soil, may also be mentioned. These young plants may be protected before transplantation by a total or partial treatment by immersion.

Embodiments described herein relate to methods of delivering to plants a treatment effective dose of agricultural compositions that include a plant viral nanoparticle (VNP) or plant virus-like particle (VLP) and at least one agrochemical agent, where the dose is determined using a computational model. The delivery methods include the steps of determining a dose of the agricultural composition required to deliver a treatment effective amount of at least one agrochemical agent to a target soil depth using a computational model; and applying the determined dose of the agricultural composition to the plant.

As shown in the Example, computational models used to determine treatment effective doses of agricultural compositions for delivery to plants can increase the application efficacy of agrochemical agents included in the composition. It was further shown that VNP or VLP carriers can deliver determined doses of agrochemical agents to targeted soil depths, e.g., depths ranging from near the surface of the soil to the plant rhizosphere.

Agricultural compositions that include agrochemical agent loaded plant VNPs and/or VLPs have shown enhanced penetration through soil allowing agents to better reach pests compared to agents administered alone. The agricultural compositions described herein have significantly greater surface area than a typical agrochemical agent does alone, thereby increasing their interaction with pests at targeted soil depths at lower doses. It was shown that the computational model can be used to predict the transport behavior (e.g., soil mobility) of an agrochemical agent associated with plant VNP or VLP in an agricultural composition, thereby allowing for the determination of the dose required to deliver a treatment effective amount of the at least one agrochemical agent to a target soil depth. It is contemplated that the determination and application of treatment effective doses of agricultural compositions described herein can reduce the risk of residual agrochemicals being released in the environment due to their over application.

In some embodiments, the computational model can be defined by a set of one or more parameters. In some embodiments, the set of parameters can include one or more of the following parameters: (a) a dispersion constant of the plant viral nanoparticle (VNP) or plant virus-like particle (VLP) through soil; (b) a dispersion constant of the agrochemical agent through soil; (c) a rate constant of VNP or VLP absorption to soil; (d) a rate constant of agrochemical absorption to soil; and (e) a rate constant of pesticide release from a VNP or VLP in fluid.

In some embodiments, the dose of an agricultural composition determined using the computational model is the amount of the agricultural composition applied to a plant that is required to deliver a treatment effective amount of the at least one agrochemical agent to a targeted depth of soil. In some embodiments, the determined dose can include the amount by weight of the agricultural composition, or the amount by weight of the agrochemical agent included in the composition, required to deliver a treatment effective amount of the at least one agrochemical agent to a targeted depth of soil. In some embodiments, the determined dose can include the amount (e.g., by weight or volume) of a given concentration of an agricultural composition in solution that is required to deliver a treatment effective amount of the at least one agrochemical agent to a targeted depth of soil. In other embodiments, the determined dose can include the amount of time a particular concentration of an agricultural composition in a solution is to be applied to a plant that is required to deliver a treatment effective amount of the at least one agrochemical agent to a targeted depth of soil.

In certain embodiments, the dose determined by the computational model can include the amount of agricultural composition that is required to maintain a desired concentration of a given agrochemical agent at a target soil depth beneath the surface for a given time. In an exemplary embodiment, the computational model can be used to determine the amount of an agricultural composition must be applied to maintain the IC₅₀ concentration of an agrochemical agent to the root level or rhizosphere of a plant for at least 24 hours.

In accordance with a method described herein, a determined dose of agricultural compositions is the amount required to deliver the dose to a target soil depth. In particular embodiments, the target soil depth can be about 0 cm to about 100 cm below the surface. In particular embodiments, the target soil depth can be about 1 cm to about 50 cm below the surface. In other embodiments, the target soil depth can be about 4 cm to about 30 cm below the surface.

In some embodiments, the target soil depth can include a depth in the soil surface region of soil. Thus, in particular embodiments, the target soil depth can range from the soil surface to a depth of about 4 cm from the surface. In some embodiments, the target depth can include the root level depth or the depth occupied by the rhizosphere of a particular plant species. As used herein, the term “rhizosphere” is the nutrient-rich region of soil immediately surrounding the plant roots governed by complex interactions between plants and the organisms that are in close association with the root.

Agricultural compositions for use in a method described herein include VNPs or and/or VLPs thereof that are used as carriers to deliver agrochemical agents to a plant. VNPs and/or VLPs can provide an economically and environmentally viable alternative to conventional synthetic nanoparticles. Plant VNPs and/or VLPs can be produced in large quantities in a short time for a relatively low price. In addition, plant VNPs and/or VLPs are exceptionally robust to the harsh environment of crop fields, biodegradable, as well as biocompatible and noninfectious, making them safe to use on industrial crops.

As shown in the Example, it was found that the use of plant VNPs or VLPs thereof can significantly enhance soil mobility of an associated agrochemical. It was further shown that the association (i.e., conjugation and/or encapsulation) of agrochemicals with plant VNPs or VLPs can prevent premature degradation of the active agrochemical agent. Therefore, in some embodiments, the plant VNPs and/or VLPs for use in a delivery method described herein can improve the biodegradability, stability, permeability, soil mobility, and/or dispersion of the at least one agrochemical agent in soil.

In some embodiments, agricultural compositions can be derived from plant viruses in the form of VNPs, which include a virus genome and are potentially infectious. However, in preferred embodiments, plant VNPs are nonpathogenic plant virus particles. In other embodiments, plant viral carriers of agrochemical agents for use in an agricultural composition described herein can be derived from plant viruses in the form of VLPs, which do not carry nucleic acid and are therefore non-infectious.

In some embodiments, a wild-type virus used for the production of the empty VLPs can be obtained according to various methods known to those skilled in the art. In embodiments where plant virus particles are used, the virus particles can be obtained from the extract of a plant infected by the plant virus. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant virus particles, such as picornavirus particles, using extraction of an infected plant are known to those skilled in the art. See Wellink J., Meth Mol Biol, 8, 205-209 (1998). Procedures are also available for obtaining virus-like particles. Saunders et al., Virology, 393(2):329-37 (2009). The disclosures of both of these references are incorporated herein by reference.

Plant VNPs and/or VLPs that are used as carriers to deliver the agrochemical agents in an agricultural composition described herein can be derived from plant virus having various geometries, such as, but not limited to, rod-shaped, spherical and icosahedral shaped plant virus.

In some embodiments, the plant VNPs and/or VLPs for use in an agricultural composition described herein include rod-shaped VNPs and/or VLPs thereof. Advantageously, rod-shaped VNPs and/or VLPs, in comparison to VNPs and/or VLPs having other geometries, such as spherical or icosahedral VNPs and/or VLPs, can provide higher loading and delivery of agrochemical agents.

The rod-shaped plant viruses used as the rod-shaped VNPs and/or VLPs can be shaped as a rigid helical rod with a helical symmetry. Rod-shaped plant VNPs and/or VLPs are distinguished from filamentous plant virus particles as being inflexible, shorter, and thicker in diameter. The rod-shaped plant VNPs and/or VLPs can have an exterior surface and an interior surface that extend from a first end to a second of the rod-shaped VNP and/or VLP. The interior surface can define a central hollow channel that extends through rod-shaped VNP and/or VLP from the first end to the second end. The channel can include the viral genome (e.g., VNP) or be substantially free of or lack the viral genome (e.g., VLP).

The rod-shaped plant virus can belong to a specific virus family, genus, or species. In some embodiments, the rod-shaped plant virus belongs to the Virgaviridae family Virgaviridae viruses have a length of about 200 to about 400 nm, and a diameter of about 15-25 nm. Virgaviridae viruses have other characteristics, such as having a single-stranded RNA positive sense genome with a 3′-tRNA like structure and no polyA tail, and coat proteins of 19-24 kilodaltons. The Virgaviridae family includes the genus Furovirus, Hordevirus, Pecluvirus, Pomovirus, Tobamovirus, and Tobravirus. In other embodiments, the rod-shaped plant virus belongs to the genus Tobamovirus. In some embodiments, the rod-shaped plant virus can include a tobamoviruses such as, but not limited to, a Paprika mild mottle virus (PaMMV), Pepper mild mottle virus (PMMoV), Ribgrass mosaic virus (RMV), Tobacco mild green mosaic virus (TMGMV), Tobacco mosaic virus (TMV), and Tomato mosaic virus (ToMV).

In some embodiments, the rod-shaped plant virus used as rod-shaped VNP and/or VLP belongs to the TMGMV species. TMGMV self assembles into a 300×18 nm rod-shaped virus with a 4 nm wide hollow interior channel Similar to TMV, TMGMV includes a single copy of coat protein (CP) arranged helically around a single stranded RNA genome. Advantageously, TMGMV also has a high surface area (3.6×10⁻¹⁴ m² on the exterior and 7.6×10⁻¹⁵ m² on the interior) compared to icosahedral viruses that can allow for higher payload delivery of agrochemical agents.

TMGMV is commercially available under the tradename Solvinix from BioProdex. It is currently EPA approved as an herbicide in the state of Florida for the treatment of the invasive weed tropical soda apple. Advantageously, TMGMV is not transmitted by insects, pollen, or other vectors; it is not seed borne and cannot self-disseminate. While TMGMV is capable of infecting solanaceous plants (e.g., tomatoes, chili peppers, and eggplants), TMGMV is unable to penetrate and infect healthy plants in the absence of a lesion wound. Furthermore, Solvinix was tested on 435 plants representing 311 species, among which only 8% of plants were killed. TMGMV can, therefore, be used as a carrier for an agrochemical agent and be applied for agricultural applications with little to no risk to the environment or the crop itself.

In other embodiments, the plant VNPs and/or VLPs that are used as carriers to deliver the agrochemical agents in an agricultural composition described herein include icosahedral shaped plant VNPs and/or VLPs thereof. Exemplary icosahedral plant viruses include the virus families Geminiviridae, Luteoviridae, Bromoviridae, Phycodnaviridae, Tymoviridae and Picornaviridae.

In some embodiments, the icosahedral plant virus is from the family Tymoviridae. In some embodiments, the Tymoviridae VNP or VLP is a Tymovirus VNP or VLP derived from a virus of the Tymovirus genus. Tymovirus virus is a virus that primarily infects plants and has a non-enveloped icosahedral and isometric structure. The diameter of a Tymovirus, such as PhMV, is about 30 nm. Use of a Tymovirus virus or Tymovirus VLP as described herein provides the advantages of improved physical stability (e.g., after cargo loading as well as in storage) and production consistency.

A Tymovirus virus can be selected from a group consisting of Physalis Mottle Virus (PhMV), Belladonna Mottle Virus, Turnip Yellow Mosaic Virus, Cacao Yellow Mosaic Virus, Clitoria Yellow Vein Virus, Desmodium Yellow Mottle Virus, Eggplant Mosiac Virus and Passion Fruit Yellow Mosaic Virus. A comparison of coat protein sequence of PhMV with other tymoviruses revealed that PhMV has a 52% identity with belladonna mottle virus (E) and 33% identity with turnip yellow mosaic virus (TYMV), showing that PhMV (previously named as belladonna mottle virus I) is a distinct Tymovirus.

In an exemplary embodiment, the icosahedral shaped plant virus used as an icosahedral shaped VNP and/or VLP belongs to the PhMV species. PhMV is a small spherical plant virus of the Tymovirus genus of positive-stranded RNA viruses. The nucleotide sequence coding for the (PhMV) coat protein was identified from the GenBank having EMBL accession number S97776 (Jocob et al., 1992). The positive-sense RNA genome is encapsidated in a protein shell consisting of 180 identical copies of coat protein (CP) arranged with T=3 icosahedral symmetry. The multiple copies of the asymmetric unit provide regularly spaced attachment sites on both the internal and external surfaces of the PhMV capsid allowing for modification of PhMV with diagnostic and therapeutic agents described herein.

The coat protein of a Tymovirus virus for use as a VLP can be synthetically produced using methods well known in the art. Methods of producing Tymovirus VLPs can include the steps of: (a) producing a recombinant polynucleotide sequence, (b) constructing a recombinant vector comprising a regulatory sequence and the recombinant polynucleotide sequence of step (a), (c) transforming a host cell with the recombinant vector of step (b) to produce a recombinant host cell, (d) growing the recombinant host cell of step (c) to produce Tymovirus virus-like particles, and (e) purifying the Tymovirus virus-like particles of step (d). The recombinant vector can further include a regulatory sequence. Exemplary regulatory sequence can include T7, SP6 and T3 promoters.

Tymovirus-derived VLPs can be formed from Tymovirus structural proteins encoded by a recombinant polynucleotide sequence that are expressed in an Escherichia coli, yeast or baculovirus heterologous expression system. In some embodiments, the heterologous expression system is an E. coli expression system. The E. coli strain can be selected from the group consisting of JM101, DH5α, BL21, HB101, BL21(DE3) pLys S, XL-1 Blue and Rossetta. In some embodiments, the recombinant polynucleotide sequence can include, for example, a nucleotide sequence encoding all, or a truncated portion, of the PhMV coat protein.

In some embodiments, the icosahedral plant virus is from the family Picornaviridae. Plant picornaviruses are relatively small, non-enveloped, positive-stranded RNA viruses with an icosahedral capsid. Plant picornaviruses have a number of additional properties that distinguish them from other picornaviruses, and are categorized as the subfamily secoviridae. In some embodiments, the plant virus is selected from the Comovirinae virus subfamily. Examples of viruses from the Comovirinae subfamily include cowpea mosaic virus, Broad bean wilt virus 1, and Tobacco ringspot virus. In a particular embodiment, the VNPs and/or VLPs of an agricultural composition described herein are derived from virus of the genus Comovirus. In some embodiments, the comovirus is a cowpea mosaic virus (CPMV).

The at least one agrochemical agent of an agricultural composition described herein can include any agrochemical agent that can be associated with plant VNPs and/or VLPs via adsorption, encapsulation and/or covalent or non-covalent conjugation, and/or agrochemical agents that are suitable for agricultural applications. Examples of agrochemical agents for use in an agricultural composition described herein include, but are not limited to, pesticides (e.g., nematicides, insecticides, acaricides, fungicides, herbicides, etc.) plant growth regulators, nutrients, pest repellents, and the like. Examples of agrochemical agents that can be included in an agricultural composition are described in U.S. Patent Application No. 2011/0200571; U.S. Pat. Nos. 8,119,150; 7,836,630; 6,776,996; 6,660,690; 6,638,994; and 6,602,823, the disclosures of which agrochemical agents found therein are incorporated by reference herein in their entirety. Particular examples include but are not limited to those discussed in greater detail below.

In some embodiments, the agrochemical agent can be a nematicide. Examples of nematicides that can be associated with a plant VNP and/or VLP in an agricultural composition described herein include, but are not limited to, anthelmintics, such as crystal violet (hexamethyl parparosaniline chloride), antibiotic nematicides, such as abamectin; carbamate nematicides, such as benomyl, carbofuran, carbosulfan, and cleothocard; oxime carbamate nematicides, such as alanycarb, aldicarb, aldoxycarb, oxamyl; organophosphorous nematicides, such as diamidafos, fenamiphos, fosthietan, phosphamidon, cadusafos, chlorpyrifos, dichlofenthion, dimethoate, ethoprophos, fensulfothion, fosthiazate, heterophos, isamidofos, isazofos, methomyl, phorate, phosphocarb, terbufos, thiodicarb, thionazin, triazophos, imicyafos, and mecarphon. Other compounds with nematicidal activity include acetoprole, benclothiaz, chloropicrin, dazomet, DB CP, DCIP, 1,2-dichloropropane, 1,3-dichloropropene, furfural, iodomethane, metam, methyl bromide, methyl isothiocyanate, and xylenols.

In other embodiments, the agrochemical agent can be a fungicide. Examples of fungicides that can be associated with a plant VNP and/or VLP in an agricultural composition described herein include, but are not limited to, aldimorph, ampropylfos, ampropylfos potassium, andoprim, anilazine, azaconazole, azoxystrobin, benalaxyl, benodanil, benomyl, benzamacril, benzamacryl-isobutyl, bialaphos, binapacryl, biphenyl, bitertanol, blasticidin-S, boscalid, bromuconazole, bupirimate, buthiobate, calcium polysulphide, capsimycin, captafol, captan, carbendazim, carboxin, carvon, quinomethionate, chiobenthiazone, chlorfenazol, chloroneb, chloropicrin, chlorothalonil, chlozolinate, clozylacon, cufraneb, cymoxanil, cyproconazole, cyprodinil, cyprofuram, debacarb, dichlorophen, diclobutrazole, diclofluanid, diclomezine, dicloran, diethofencarb, difenoconazole, dimethirimol, dimethomorph, dimoxystrobin, diniconazole, diniconazole-M, dinocap, diphenylamine, dipyrithione, ditalimfos, dithianon, dodemorph, dodine, drazoxolon, edifenphos, epoxiconazole, etaconazole, ethirimol, etridiazole, famoxadon, fenapanil, fenarimol, fenbuconazole, fenfuram, fenitropan, fenpiclonil, fenpropidin, fenpropimorph, fentin acetate, fentin hydroxide, ferbam, ferimzone, fluazinam, fludioxonil, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox, guazatine, hexachlorobenzene, hexaconazole, hymexazole, imazalil, imibenconazole, iminoctadine, iminoctadine albesilate, iminoctadine triacetate, iodocarb, ipconazole, iprobenfos (IBP), iprodione, irumamycin, isoprothiolane, isovaledione, kasugamycin, kresoxim-methyl, copper preparations, such as: copper hydroxide, copper naphthenate, copper oxychloride, copper sulphate, copper oxide, oxine-copper and Bordeaux mixture, mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin, nickel dimethyldithiocarbamate, nitrothal-isopropyl, nuarimol, ofurace, oxadixyl, oxamocarb, oxolinic acid, oxycarboxim, oxyfenthiin, paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, prothiocinazole, pyraclostrobin, pyrazophos, pyrifenox, pyrimethanil, pyroquilon, pyroxyfur, quinconazole, quintozene (PCNB), sulphur and sulphur preparations, tebuconazole, tecloftalam, tecnazene, tetcyclasis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole, uniconazole, validamycin A, vinclozolin, viniconazole, zarilamide, zineb, ziram and also Dagger G, OK-8705, OK-8801, .alpha.-(1,1-dimethylethyl)-.beta.-(2-phenoxyethyl)-1H-1,2,4-tri-azole-1-e-thanol, .alpha.-(2,4-dichlorophenyl)-.beta.-fluoro-.beta.-propyl-1H-1,2,4-triazol-e-1-ethanol, .alpha.-(2,4-dichlorophenyl)-.beta.-methoxy-.alpha.-methyl-1H-1,2,4-triaz-ole-1-ethanol, .alpha.-(5-methyl-1,3-dioxan-5-yl)-.beta.-[[4-(trifluoromethyl)-phenyl]-m-ethylene]-1H-1,2,4-triazole-1-ethanol, (5RS,6RS)-6-hydroxy-2,2,7,7-tetramethyl-5-(1H-1,2,4-triazol-1-yl)-3-octan-one, (E)-.alpha.-(methoxyimino)-N-methyl-2-phenoxy-phenylacetamide, 1-isopropyl {2-methyl-1-[[[1-(4-methylphenyl)-ethyl]-amino]-carbonyl]-prop-yl}carbama-te, 1-(2,4-dichlorophenyl)-2-(1H-1,2,4-triazol-1-yl)-ethanone-O-(phenylmet-hyl-)-oxime, 1-(2-methyl-1-naphthalenyl)-1H-pyrrole-2,5-dione, 1-(3,5-dichlorophenyl)-3-(2-propenyl)-2,5-pyrrolidindione, 1-[(diiodomethyl)-sulphonyl]-4-methyl-benzene, 1-[[2-(2,4-dichlorophenyl)-1,3-dioxolan-2-yf]-methyl]-1H-imidazole, 1-[[2-(4-chlorophenyl)-3-phenyloxiranyl]-methyl]-1H-1,2,4-triazole, 1-[1-[2-[(2,4-dichlorophenyl)-methoxy]-phenyl]-ethenyl]-1H-imidazole, 1-methyl-5-nonyl-2-(phenylmethyl)-3-pyrrolidinole, 2′,6′-dibromo-2-methyl-4′-trifluoromethoxy-4′-trifluoro-methyl-1,3-thiazo-le-5-carboxanilide, 2,2-dichloro-N-[1-(4-chlorophenyl)-ethyl]-1-ethyl-3-methyl-cyclopropanecarboxamide, 2,6-dichloro-5-(methylthio)-4-pyrimidinyl-thiocyanate, 2,6-dichloro-N-(4-trifluoromethylbenzyl)-benzamide, 2,6-dichloro-N-[[4-(trifluoromethyl)-phenyl]-methyl]-benzamide, 2-(2,3,3-triiodo-2-propenyl)-2H-tetrazole, 2-[(1-methylethyl)-sulphonyl]-5-(trichloromethyl)-1,3,4-thiadiazole, 2-[[6-deoxy-4-O-(4-O-methyl-D-glycopyranosyl)-.alpha.-D-glucopyran-osyl]-amino]-4-methoxy-1H-pyrrolo[2,3-d]pyrimidine-5-carbonitrile, 2-aminobutane, 2-bromo-2-(bromomethyl)-pentanedinitrile, 2-chloro-N-(2,3-dihydro-1,1,3-trimethyl-1H-inden-4-yl)-3-pyridinecarboxamide, 2-chloro-N-(2,6-dimethylphenyl)-N-(isothiocyanatomethyl)-acetamide, 2-phenylphenol (OPP), 3,4-dichloro-1-[4-(difluoromethoxy)-phenyl]-1H-pyrrole-2,5-dione, 3,5-dichloro-N-[cyano[(1-methyl-2-propynyl)-oxy]-methyl]-benzamide, 3-(1,1-dimethylpropyl-1-oxo-1H-indene-2-carbonitrile, 3-[2-(4-chlorophenyl)-5-ethoxy-3-isoxazolidinyl]-pyridine, 4-chloro-2-cyano-N,N-dimethyl-5-(4-methylphenyl)-1H-imidazole-1-sulphonamide, 4-methyl-tetrazolo[1,5-a]quinazolin-5(4H)-one, 8-(1,1-dimethylethyl)-N-ethyl-N-propyl-1,4-dioxaspiro[4,5]decane-2-methanamine, 8-hydroxyquinoline sulphate, 9H-xanthene-2-[(phenylamine)-carbonyl]-9-carboxylic hydrazide, bis-(1-methylethyl)-3-methyl-4-[(3-methylbenzoyl)-oxy]-2,5-thiophenedicarboxylate, cis-1-(4-chlorophenyl)-2-(1H-1,2,4-triazol-1-yl)-cycloheptanol, cis-4-[3-[4-(1,1-dimethylpropyl)-phenyl-2-methylpropyl]-2,6-dimethyl-morp-holine hydrochloride, ethyl [(4-chlorophenyl)-azo]-cyanoacetate, potassium bicarbonate, methanetetrathiol-sodium salt, methyl 1-(2,3-dihydro-2,2-dimethyl-1H-inden-1-yl)-1H-imidazole-5-carboxylate, methyl N-(2,6-dimethylphenyl)-N-(5-isoxazolylcarbonyl)-DL-alaninate, methyl N-(chloroacetyl)-N-(2,6-dimethylphenyl)-DL-alaninate, N-(2,3-dichloro hydroxyphenyl)-1-methyl-cyclohexanecarboxamide, N-(2,6-dimethylphenyl)-2-methoxy-N-(tetrahydro-2-oxo-3-furanyl)-acetamide-dimethylphenyl)-2-methoxy-N-(tetrahydro-2-oxo-3-thienyl)-acetamid-e, N-(2-chloro-4-nitrophenyl)-4-methyl-3-nitro-benzenesulphonamide, N-(4-cyclohexylphenyl)-1,4,5,6-tetrahydro-2-pyrimidinamine, N-(4-hexylphenyl)-1,4,5,6-tetrahydro-2-pyrimidinamine, N-(5-chloro-2-methylphenyl)-2-methoxy-N-(2-oxo-3-oxazolidinyl)-acetamide, N-(6-methoxy)-3-pyridinyl)-cyclopropanecarboxamide, N-[2,2,2-trichloro-1-[(chloro acetyl)-amino]-ethyl]-benzamide, N-[3-chloro-4,5-bis(2-propinyloxy)-phenyl]-N-methoxy-methanimidamide, N-formyl-N-hydroxy-DL-alanine-sodium salt, O,O-diethyl [2-(dipropylamino)-2-oxoethyl]-ethylphosphoramidothioate, O-methyl S-phenyl phenylpropylphosphoramidothioate, S-methyl 1,2,3-benzothiadiazole-7-carbothioate, spiro[2H]-1-benzopyrane-2,1′(3′H)-isobenzofuran]-3′-one, and Trilex-Yield Shield (Bayer CropScience) alone or in combination.

In still other embodiments, the agrochemical agent can be an insecticide.

Examples of insecticides that can be conjugated to and/or loaded on the interior and/or exterior surface of the rod-shaped VNP and/or VLP include, but are not limited to, neonicotinoid insecticides such 1-(6-chloro-3-pyridylmethyl)-N-nitroimidazolidin-2-ylideneamine (imidacloprid), 3-(6-chloro-3-pyridylmethyl)-1,3-thiazolidin-2-ylidene cyanamide (thiacloprid), 1-(2-chloro-1,3-thiazol-5-ylmethyl)-3-methyl-2-nitroguanidine (clothianidin), nitempyran, N¹-[(6-chloro-3-pyridyl)methyl]-N.sup.2-cyano-N¹-methylacetamidine (acetamiprid), 3-(2-chloro-1,3-thiazol-5-ylmethyl)-5-methyl-1,3,5-oxadiazinan-4-ylidene(-nitro)amine (thiamethoxam) and 1-methyl-2-nitro-3-(tetrahydro-3-furylmethyl) guanidine (dinotefuran).

In other embodiments, the agrochemical agent can be a herbicide. Examples of herbicides that can be associated with a plant VNP and/or VLP in an agricultural composition described herein include, but are not limited to: amide herbicides such as allidochlor, beflubutamid, benzadox, benzipram, bromobutide, cafenstrole, CDEA, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid and tebutam; anilide herbicides such as chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen and propanil; arylalanine herbicides such as benzoylprop, flampropand flamprop-M; chloroacetanilide herbicides such as acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor and xylachlor; sulfonanilide herbicides such as benzofluor, perfluidone, pyrimisulfan and profluazol; sulfonamide herbicides such as asulam, carbasulam, fenasulam and oryzalin; antibiotic herbicides such as bilanafos; benzoic acid herbicides such as chloramben, dicamba, 2,3,6-TBA and tricamba; pyrimidinyloxybenzoic acid herbicides such as bispyribac and pyriminobac; pyrimidinylthiobenzoic acid herbicides such as pyrithiobac; phthalic acid herbicides such as chlorthal; picolinic acid herbicides such as aminopyralid, clopyralid and picloram; quinolinecarboxylic acid herbicides such as quinclorac and quinmerac; arsenical herbicides such as cacodylic acid, CMA, DSMA, hexaflurate, MAA, MAMA, MSMA, potassium arsenite and sodium arsenite; benzoylcyclohexanedione herbicides such as mesotrione, sulcotrione, tefuryltrione and tembotrione; benzofuranyl alkylsulfonate herbicides such as benfuresate and ethofumesate; carbamate herbicides such as asulam, carboxazole chlorprocarb, dichlormate, fenasulam, karbutilate and terbucarb; carbanilate herbicides such as barban, BCPC, carbasulam, carbetamide, CEPC, chlorbufam, chlorpropham, CPPC, desmedipham, phenisopham, phenmedipham, phenmedipham-ethyl, propham and swep; cyclohexene oxime herbicides such as alloxydim, butroxydim, clethodim, cloproxydim, cycloxydim, profoxydim, sethoxydim, tepraloxydim and tralkoxydim; cyclopropylisoxazole herbicides such as isoxachlortole and isoxaflutole; dicarboximide herbicides such as benzfendizone, cinidon-ethyl, flumezin, flumiclorac, flumioxazin and flumipropyn; dinitroaniline herbicides such as benfluralin, butralin, dinitramine, ethalfluralin, fluchloralin, isopropalin, methalpropalin, nitralin, oryzalin, pendimethalin, prodiamine, profluralin and trifluralin; dinitrophenol herbicides such as dinofenate, dinoprop, dinosam, dinoseb, dinoterb, DNOC, etinofen and medinoterb; diphenyl ether herbicides such as ethoxyfen; nitrophenyl ether herbicides such as acifluorfen, aclonifen, bifenox, chlomethoxyfen, chlomitrofen, etnipromid, fluorodifen, fluoroglycofen, fluoronitrofen, fomesafen, furyloxyfen, halosafen, lactofen, nitrofen, nitrofluorfen and oxyfluorfen; dithiocarbamate herbicides such as dazomet and metam; halogenated aliphatic herbicides such as alorac, chloropon, dalapon, flupropanate, hexachloroacetone, iodomethane, methyl bromide, monochloroacetic acid, SMA and TCA; imidazolinone herbicides such as imazamethabenz, imazamox, imazapic, imazapyr, imazaquin and imazethapyr; inorganic herbicides such as ammonium sulfamate, borax, calcium chlorate, copper sulfate, ferrous sulfate, potassium azide, potassium cyanate, sodium azide, sodium chlorate and sulfuric acid; nitrile herbicides such as bromobonil, bromoxynil, chloroxynil, dichlobenil, iodobonil, ioxynil and pyraclonil; organophosphorus herbicides such as amiprofos-methyl, anilofos, bensulide, bilanafos, butamifos, 2,4-DEP, DMPA, EBEP, fosamine, glufosinate, glyphosate and piperophos; phenoxy herbicides such as bromofenoxim, clomeprop, 2,4-DEB, 2,4-DEP, difenopenten, disul, erbon, etnipromid, fenteracol and trifopsime; phenoxyacetic herbicides such as 4-CPA, 2,4-D, 3,4-DA, MCPA, MCPA-thioethyl and 2,4,5-T; phenoxybutyric herbicides such as 4-CPB, 2,4-DB, 3,4-DB, MCPB and 2,4,5-TB; phenoxypropionic herbicides such as cloprop, 4-CPP, dichlorprop, dichlorprop-P, 3,4-DP, fenoprop, mecopropand mecoprop-P; aryloxyphenoxypropionic herbicides such as chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P and trifop; phenylenediamine herbicides such as dinitramine and prodiamine; pyrazolyl herbicides such as benzofenap, pyrazolynate, pyrasulfotole, pyrazoxyfen, pyroxasulfone and topramezone; pyrazolylphenyl herbicides such as fluazolate and pyraflufen; pyridazine herbicides such as credazine, pyridafol and pyridate; pyridazinone herbicides such as brompyrazon, chloridazon, dimidazon, flufenpyr, metflurazon, norflurazon, oxapyrazon and pydanon; pyridine herbicides such as aminopyralid, cliodinate, clopyralid, dithiopyr, fluoroxypyr, haloxydine, picloram, picolinafen, pyriclor, thiazopyr and triclopyr; pyrimidinediamine herbicides such as iprymidam and tioclorim; quaternary ammonium herbicides such as cyperquat, diethamquat, difenzoquat, diquat, morfamquat and paraquat; thiocarbamate herbicides such as butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate and vernolate; thiocarbonate herbicides such as dimexano, EXD and proxan; thiourea herbicides such as methiuron; triazine herbicides such as dipropetryn, triaziflam and trihydroxytriazine; chlorotriazine herbicides such as atrazine, chlorazine, cyanazine, cyprazine, eglinazine, ipazine, mesoprazine, procyazine, proglinazine, propazine, sebuthylazine, simazine, terbuthylazine and trietazine; methoxytriazine herbicides such as atraton, methometon, prometon, secbumeton, simeton and terbumeton; methylthiotriazine herbicides such as ametryn, aziprotryne, cyanatryn, desmetryn, dimethametryn, methoprotryne, prometryn, simetryn and terbutryn; triazinone herbicides such as ametridione, amibuzin, hexazinone, isomethiozin, metamitron and metribuzin; triazole herbicides such as amitrole, cafenstrole, epronaz and flupoxam; triazolone herbicides such as amicarbazone, bencarbazone, carfentrazone, flucarbazone, propoxycarbazone, sulfentrazone and thiencarbazone-methyl; triazolopyrimidine herbicides such as cloransulam, diclosulam, florasulam, flumetsulam, metosulam, penoxsulam and pyroxsulam; uracil herbicides such as butafenacil, bromacil, flupropacil, isocil, lenacil and terbacil; 3-phenyluracils; urea herbicides such as benzthiazuron, cumyluron, cycluron, dichloralurea, diflufenzopyr, isonoruron, isouron, methabenzthiazuron, monisouron, noruron and saflufenacil; phenylurea herbicides such as anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluoron, phenobenzuron, siduron, tetrafluoron and thidiazuron; pyrimidinylsulfonylurea herbicides such as amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron and trifloxysulfuron; triazinylsulfonylurea herbicides such as chlorsulfuron, cinosulfuron, ethametsulfuron, iodosulfuron, metsulfuron, prosulfuron, thifensulfuron, triasulfuron, tribenuron, triflusulfuron and tritosulfuron; thiadiazolylurea herbicides such as buthiuron, ethidimuron, tebuthiuron, thiazafluoron and thidiazuron; and unclassified herbicides such as acrolein, allyl alcohol, aminocyclopyrachlor, azafenidin, benazolin, bentazone, benzobicyclon, buthidazole, calcium cyanamide, cambendichlor, chlorfenac, chlorfenprop, chlorflurazole, chlorflurenol, cinmethylin, clomazone, CPMF, cresol, ortho-dichlorobenzene, dimepiperate, endothal, fluoromidine, fluridone, fluorochloridone, flurtamone, fluthiacet, indanofan, indaziflam, methazole, methyl isothiocyanate, nipyraclofen, OCH, oxadiargyl, oxadiazon, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan and tritac.

In yet other embodiments, the agrochemical agent can be a plant growth regulator. In some embodiments, the plant growth regulator can include a plant growth promoter. A plant growth promoter can include any agrochemical agent capable of promoting cell division, cell enlargement, flowering, fruiting and/or seed formation. Exemplary plant growth promoters can include, but are not limited to auxins, gibberellins and cytokinins.

In some embodiments, the plant growth regulator can include a plant growth Inhibitor. A plant growth inhibitor can include any agrochemical agent capable of inhibiting growth and/or promoting dormancy and abscission in plants. Exemplary plant growth inhibitors can include, but are not limited to, an abscisic acid, maleic hydrazide, naphthalene methyl acetate, 6-benzylaminopurines, brassinosteroid, ammonia oxygen Ethyl vinyl glycine and multiple-effect azole plant growth regulators.

Additional examples of plant growth regulators that can be associated with a plant VNP and/or VLP in an agricultural composition described herein include but not limited to azoles (such as uniconazole, and paclobutrazol), cyclohexane carboxylates (such as trinexapac-ethyl, and prohexadione-calcium), pyrimidinyl carbinols (such as flurprimidol, and ancymidol), quarternary ammoniums (such as chlormequat-chloride, and mepiquat-chloride), and sulphonyl-amino phenyl-acetamides (such as mefluidide), and those described in PCT Patent Application WO 2011063947.

Plant VNPs and/or VLPs can be associated with agrochemical agents in agricultural compositions described herein through adsorption, encapsulation and/or conjugation. The agrochemical agents can be conjugated to and/or loaded on the interior and/or exterior surface of the VNPs and/or VLPs by any suitable technique. The term “conjugating” when made in reference to an agrochemical agent and a VNP and/or VLP as used herein includes covalently or non-covalently linking, attaching, binding, and/or coupling the agent to the VNPs and/or VLPs. The agrochemical agent can be covalently or non-covalently linked to the interior or the exterior surfaces of the VNPs and/or VLPs or to both the interior and the exterior surface of the VNPs and/or VLPs. The location of the agrochemical agent on the interior or exterior can be governed by the amino acids of the viral coat protein that are selected as reactive sites for covalent linking or the electrostatic properties of the exposed amino acid residues of the interior and/or exterior surface for non-covalent linking.

In some embodiments, plant VNPs or VLPs can be associated with at least one agrochemical agent by loading with or conjugation to the agrochemical agent through the use of non-covalent infusion techniques that facilitate efficient cargo loading of an agrochemical agent into the plant VNPs or VLPs. In some embodiments, rather than covalent attachment, at least one agrochemical agent described herein can be loaded on an exterior and/or interior surface of the plant VNPs and/or VLPs in a non-covalent manner by associating them with the plant VNPs and/or VLPs. The agrochemical agent can associate with the plant VNPs as a result of the affinity of the agrochemical agent to an exposed chemical group of the amino residue of the coat protein. Affinity is the tendency of a compound to naturally associate with another object. Affinity is influenced by non-covalent intermolecular interactions between the compound and the object, such as hydrogen bonding, electrostatic interactions, hydrophobic interactions, and Van der Waals forces.

In one example, positively charged agrochemical agents can have an affinity via electrostatic interactions to negatively charged interior or exterior surfaces of the plant VNPs and/or VLPs. The negatively charged interior or exterior surfaces can be provided by negatively charged amino acid residues, charged groups, polymers, and/or dendrimers on the interior and/or exterior surface of the plant VNPs and/or VLPs that are intrinsic to the VNPs and/or VLPs and/or provide by chemical modification and/or genetic addition to the plant VNPs and/or VLPs. By way of example, carboxylate groups of exposed aspartic acid and glutamic acid residues on the interior and exterior surface of the plant VNPs and/or VLPs can provide a negatively charged group that can interact electrostatically with the positively charged agrochemical agent.

It will be appreciated that the affinity of an agrochemical agent for the interior and/or exterior surface of the VNP and/or VLP can be readily determined. For example, gel mobility shift assays, crosslinking assays, optical absorbance and fluorescence assays, calorimetric assays, and/or surface Plasmon resonance assays to determine the association and dissociation kinetics and affinities of agrochemical agents for the plant VNPs and/or VLPs. Furthermore, any agrochemical agent exhibiting low affinity can be readily modified with a small, positively charged tag to bind to plant VNP and/or VLP.

In some embodiments, positively charged agrochemical agents can be non-covalently loaded onto negatively charged interior or exterior surfaces of the plant VNPs and/or VLPs by electrostatic interactions in a reversible manner, in order to facilitate release of the agrochemical agents from the VNPs and/or VLPs to a pest, plant, part of plant, plant organ, plant propagation material, and/or surrounding area thereof. The release rate of the agrochemical agent from the plant VNPs and/or VLPs can be controlled and be dependent on the pH of the microenvironment to which the agrochemical composition described herein is administered. Advantageously, administration of the agrochemical composition to soils having lower pH can promote more ready diffusion of the positively charged non-covalently loaded agrochemical agents from the plant VNPs and/or VLPs. As the pH of the soil decreases, a larger number of carboxylate groups can become protonated and carry a net neutral charge that can no longer interact with positively charged agrochemical agents allowing the positively charged agrochemical agents to diffuse from the plant VNPs and/or VLPs to the soil.

In some embodiments, an agrochemical agent is passively encapsulated within a plant VNPs or VLPs using non-covalent infusion. In some embodiments, agrochemical agents can be loaded with, or encapsulated, by mixing plant VNPs or VLPs with a molar excess of 500:1, 1000:1, 2000:1, 3000:1, 4000:1, 6000:1, 10,000:1, 20,000:1 of at least one agrochemical agent in a buffer. In an exemplary embodiment, an encapsulated formulation can be prepared by mixing 1 mgml⁻¹ of TMGMV, CPMV or PhMV with a 5000-fold molar excess of a pesticide in 10 mM potassium phosphate (KP) buffer (pH 7.8) overnight at room temperature with agitation.

In another exemplary embodiment, Tymovirus VNPs or Tymovirus VLPs, can be loaded with an agrochemical agent via non-covalent infusion by incubating the VNPs or VLPs in a bathing solution containing the guest molecule(s) (e.g., agrochemical agent) at a molar excesses ranging from about 100 to about 10,000 molecules per VLP) in KP buffer with 10% (v/v) DMSO overnight at room temperature. After the reaction, excess guest molecules can be removed by ultracentrifugation and the amount of guest molecule can be quantified by, for example, UV/visible spectroscopy. Differences in loading efficiency may reflect the density and distribution of charged and hydrophobic groups on the guest molecules (e.g., an agrochemical agent). For example, Tymovirus VNP or Tymovirus VLPs typically have a greater affinity for guest molecules having a positive charge.

In some embodiments, plant VNPs or VLPs can be associated with at least one agrochemical agent by conjugation to the agrochemical agent. The agrochemical agent can be conjugated to the interior and/or exterior surface of the VNP and/or VLP to provide an agricultural composition which can be readily delivered to a plant, and/or surrounding soil surface area thereof in a method described herein.

In some embodiments, agrochemical agents described herein can be covalently bound to chemically modified exposed amino acid residues on the interior and/or exterior surface of the plant VNPs and/or VLPs, such as carboxylate groups of exposed glutamic acid and aspartic acid residues on the interior and/or exterior surface of the VNPs and/or VLPs, such as rod-shaped VNPs and/or VLPs. The carboxylate groups of these amino acids also present attractive targets for functionalization using carbodiimide activated linker molecules. Exposed cysteines and lysine residues can also be present which facilitate chemical coupling via thiol-selective chemistry (e.g., maleimide-activated compounds). Further, exposed tyrosines on the on the interior and/or exterior surface of the plant VNPs and/or VLPs can be modified using diazonium coupling reactions. In addition, genetic modification can be applied to introduce any desired functional residue, including non-natural amino acids, e.g., alkyne- or azide-functional groups. See Hermanson, G. T. Bioconjugation Techniques. (Academic Press, 2008) and Pokorski, J. K. and N. F. Steinmetz, Mol Pharm 8(1): 29-43 (2011), the disclosures of which are incorporated herein by reference.

By way of example, 1-Ethyl-3-(3-dimethylaminopropyl)carbodiimide (EDC) coupling can used to chemically modifiy surface exposed glutamic and/or aspartic residues of rod-shaped VNPs and/or VLPs to introduce alkyne ligands. The introduced alkyne ligands can then be reacted with azide groups attached to agrochemical agents using Cu(I)-catalyzed alkyne-azide cycloaddition (click chemistry).

In other embodiments, a suitable chemical binder group can be used. A binder group can serve to increase the chemical reactivity of a substituent on either the agrochemical agent or plant VNP and/or VLP, and thus increase the coupling efficiency. Examples of binder chemistries include maleimidyl binders, which can be used to bind to thiol groups, isothiocyanate and succinimidyl (e.g., N-hydroxysuccinimidyl (NHS)) binders, which can bind to free amine groups, diazonium which can be used to bind to phenol, and amines, which can be used to bind with free acids such as carboxylate groups using carbodiimide activation.

Useful functional groups present on exposed viral coat proteins of the plant VNPs and/or VLPs based on the particular amino acids present, and additional groups can be designed into recombinant viral coat proteins. It will be evident to those skilled in the art that a variety of bifunctional or polyfunctional reagents, both homo- and hetero-functional (such as those described in the catalog of the Pierce Chemical Co., Rockford, Ill.), can be employed as a binder group. Coupling can be effected, for example, through amino groups, carboxyl groups, sulfhydryl groups or oxidized carbohydrate residues.

Other types of binding chemistries are also available. For example, methods for conjugating polysaccharides to peptides are exemplified by, but not limited to coupling via alpha- or epsilon-amino groups to NaIO₄-activated oligosaccharide (Bocher et al., J. Immunol. Methods 27, 191-202 (1997)), using squaric acid diester (1,2-diethoxycyclobutene-3,4-dione) as a coupling reagent (Tietze et al. Bioconjug Chem. 2:148-153 (1991)), coupling via a peptide binder wherein the polysaccharide has a reducing terminal and is free of carboxyl groups (U.S. Pat. No. 5,342,770), and coupling with a synthetic peptide carrier derived from human heat shock protein hsp65 (U.S. Pat. No. 5,736,146). Further methods for conjugating polysaccharides, proteins, and lipids to plant virus peptides are described by U.S. Pat. No. 7,666,624.

In some embodiments, the at least one agrochemical agent is conjugated to the external surface of plant VNPs and/or VLPs. In an exemplary embodiment, agrochemical agents can be conjugated to the external surface of TMGMV particles. For example, agrochemical agents can be conjugated to the external surface by two tyrosine side chains (Tyr2 and Tyr 139) of TMGMV. In other embodiments, agrochemical agents are conjugated to surface exposed lysine side chains of PMV or CPMV plant virus nanoparticles.

In some embodiments, the agrochemical agent can be indirectly conjugated to agrochemical agent via a linking molecule or a suitable chemical linker group. A linker group can serve to increase the chemical reactivity of a substituent on either the agent or the virus particle, and thus increase the coupling efficiency. A preferred group suitable for attaching agents to the plant VNPs and/or VLPs are lysine residues present in the viral coat protein.

Suitable linkage chemistries include maleimidyl linkers and alkyl halide linkers and succinimidyl (e.g., N-hydroxysuccinimidyl (NHS)) linkers (which react with a primary amine on the plant virus particle). Several primary amine and sulfhydryl groups are present on viral coat proteins, and additional groups can be designed into recombinant viral coat proteins. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be effected, for example, through amino groups, carboxyl groups, sulfhydryl groups or oxidized carbohydrate residues.

In some embodiments, it can be desirable to use a linker group which is cleavable or which is gradually cleavable over time in the environment. A number of different cleavable linker groups have been described. The mechanisms for the intracellular release of an agent from these linker groups include cleavage by reduction of a disulfide bond (e.g., U.S. Pat. No. 4,489,710); by irradiation of a photolabile bond (e.g., U.S. Pat. No. 4,625,014); by hydrolysis of derivatized amino acid side chains (e.g., U.S. Pat. No. 4,638,045); by serum complement-mediated hydrolysis (e.g., U.S. Pat. No. 4,671,958); and acid-catalyzed hydrolysis (e.g., U.S. Pat. No. 4,569,789).

In some embodiments, the linker group can include a cleavable linker where the cleavable linker is used to promote the slow and controlled release of the agrochemical, such as a pH sensitive linker group. For example, the cleavable linker can be stable enough to allow the carrier to reach the target depth in soil before the agrochemical agent cargo is dispersed, such as a labile ester linker with a half-life release of 4 days.

In other embodiments, at least two different agrochemical agents can be associated with (e.g., conjugated to and/or loaded with) separate plant VNPs and/or VLPs or the same plant VNPs and/or VLPs. The at least two different agrochemical agents can demonstrate synergistic activity compared to the activity of the individual ingredients in the combination. Each combination of agrochemical agents associated with the plant VNPs and/or VLPs may have advantageous properties for protecting plants against, for example, (i) pathogenic, such as phytopathogenic, especially fungi, attack or infestation, which result in disease and damage to the plant and/or (ii) insect or nematode attack or damage; particularly in the instance of plants, the agricultural compositions can control or prevent the pest damage on a seed, or parts of plant, plant organs and/or plants. Further, a combination according to the invention, in the absence of pathogenic or insect and/or nematode pressure, may improve the growth of a plant.

Such properties are for example the synergistically enhanced actions of combinations compared to the individual ingredients of the combination of agrochemical agents, resulting in, for example, lower pathogenic pest damage, lower rates of application, or a longer duration of action. In the instance of agriculture, the enhanced actions may show an improvement in the growing characteristics of a plant by, for example, higher than expected control of the pest damage, or higher than expected yield, stand establishment, germination, etc.

The improvement in the growing (or growth) characteristics of a plant delivered an agricultural composition in accordance with a method describe herein can manifest in a number of different ways, but will typically result in a better product of the plant. It can, for example, manifest in improving the yield and/or vigour of the plant or quality of the harvested product from the plant, which improvement may not be connected to the control of pests, such as fungi, insects and nematodes.

As used herein the phrase “improving the yield” of a plant relates to an increase in the yield of a product of the plant by a measurable amount over the yield of the same product of the plant produced under the same conditions, but without the application of the subject method. It is preferred that the yield be increased by at least about 0.5%, more preferred that the increase be at least about 1%, even more preferred is about 2%, and yet more preferred is about 4%, or more. Yield can be expressed in terms of an amount by weight or volume of a product of the plant on some basis. The basis can be expressed in terms of time, growing area, weight of plants produced, amount of a raw material used, or the like.

As used herein the phrase “improving the vigour” of a plant relates to an increase or improvement of the vigour rating, or the stand (the number of plants per unit of area), or the plant height, or the plant canopy, or the visual appearance (such as greener leaf colour), or the root rating, or emergence, or protein content, or increased tillering, or bigger leaf blade, or less dead basal leaves, or stronger tillers, or less fertilizer needed, or less seeds needed, or more productive tillers, or earlier flowering, or early grain maturity, or less plant verse (lodging), or increased shoot growth, or earlier germination, or any combination of these factors, or any other advantages familiar to a person skilled in the art, by a measurable or noticeable amount over the same factor of the plant produced under the same conditions, but without the application of the subject method.

When it is said that the present method is capable of “improving the yield and/or vigour” of a plant, the present method results in an increase in either the yield, as described above, or the vigor of the plant, as described above, or both the yield and the vigor of the plant.

A method of delivering an agricultural composition described herein includes the step of applying the determined dose of the agricultural composition to the plant. Applying the determined dose of the agricultural composition to a plant can include applying to the plant, part of plant, and to the soil surface approximate a plant. For example, the determined dose can be applied to a soil surface area corresponding to the critical root zone of the plant. The determined dose can be also be applied to a soil surface area corresponding to the total or full root zone, i.e., the maximum extent of root area of a plant.

An agricultural composition described herein can, for example, be applied to a plant and/or surrounding soil surface areas thereof directly or indirectly by any suitable technique, including but not limited to spraying, atomizing, dusting, scattering, coating or pouring, depending upon the particular plant or crop being treated. In particular embodiments, a determined dose of the agricultural composition is applied to a plant via spraying. In some embodiments, determined doses of agricultural composition may be applied to soil before planting, at the time of planting, or any time after planting.

Delivery methods of agricultural compositions described herein can be used in the agricultural sector and related fields of use for controlling or preventing damage by pests, such as insect, nematode and pathogen. Agricultural compositions described herein, especially those containing one or more pesticidal agents selected, independently from each other may be effective against pest control, such as control of pests selected from Nematoda, Insecta and Arachnida. In that instance, the combination can also be applied on the pest to control or prevent pest damage and protect the desired material (e.g., plant and part of plant) from pest damage.

Particular pests controlled by the delivery methods and compositions described herein include those from the class Nematoda, for example, the species of Tylenchus spp., Atylenchus spp., Anguina spp., Rotylenchus spp., Criconema spp., Tylenchulus spp., Paratylenchus spp., Aphenlenchus spp., Bursaphelenchus spp., Paralongidorus spp., Trichodorus spp., Meloidogyne spp. (for example, Meloidogyne incognita and Meloidogyne javanica), Heterodera spp. (for example, Heterodera glycines, Heterodera schachtii, Heterodora avenae and Heterodora trifolii), Globodera spp. (for example, Globodera rostochiensis), Radopholus spp. (for example, Radopholus similes), Rotylenchulus spp., Pratylenchus spp. (for example, Pratylenchus neglectans and Pratylenchus penetrans), Aphelenchoides spp., Helicotylenchus spp., Hoplolaimus spp., Paratrichodorus spp., Longidorus spp., Nacobbus spp., Subanguina spp. Belonolaimus spp., Criconemella spp., Criconemoides spp. Ditylenchus spp., Dolichodorus spp., Hemicriconemoides spp., Hemicycliophora spp., Hirschmaniella spp., Hypsoperine spp., Macroposthonia spp., Melinius spp., Punctodera spp., Quinisulcius spp., Scutellonema spp., Xiphinema spp., and Tylenchorhynchus spp.

The delivery methods described herein can offer opportunities to manage resistance in pests, for example, Plutella spp. as well as to proactively manage insecticide resistance in various pests.

In some embodiments, agrochemical agents included in the agricultural composition described herein, may also be effective for enhancing the plants' traits. Examples of enhanced plant traits include, but are not limited to, increased stem girth, change in leaf color, early flowering, synchronization in flowering, decrease in the lodging, control of the canopy size of a plant, delaying or eliminating tie-up of crops, increase in the disease resistance, enhancing the water utilization/improving the water use efficiency, including but not limited to decreasing the watering and/or less frequent watering (demonstrated by less wilting of the plant, the ability of the plant to rejuvenate following a suspension in watering), higher yield, higher quality/healthier plant appearance, greater transportability, decreasing the insect damage, and smaller plant canopies. Synchronized flowering is indicated by blooms materializing within 0.5 to 1 days of one another throughout the entire crop. Such a combination is particularly well suited for use for plants and propagation material thereof which are transplanted.

In an embodiment, further agent(s), such as active agrochemical agents or ingredient(s), can be used with an agricultural composition described herein. Therefore, agricultural compositions described herein may be mixed with, for example, one or more other known pesticides, such as other fungicides, insecticides, nematicides, etc. The use of additional agents, such as other active ingredients, can be for reasons, for example, broader spectrum control (e.g., wider variety of pests, diseases, etc), lower rates, synergy and economy. A skilled person would understand that a single pesticidal active ingredient may have activity in more than one area of pest control, for example, a pesticide may have fungicide, insecticide and nematicide activity. Specifically, aldicarb is known for insecticide, acaricide and nematicide activity, while metam is known for insecticide, herbicide, fungicide and nematicide activity, and thiabendazole and captan can provide nematicide and fungicide activity.

In some embodiments, agricultural compositions including the agrochemical agent(s) associated with VNPs and/or VLPs described herein can be provided as suitable formulations, such as solutions, emulsions, wettable powders, suspensions, powders, dusts, pastes, soluble powders, granules, suspoemulsion concentrates, natural and synthetic materials impregnated with active compound, and ultrafine encapsulations in polymeric materials. These formulations can produced in the known manner, for example by mixing the active compound with extenders, that is, liquid solvents and/or solid carriers, optionally with the use of surfactants, that is, emulsifiers and/or dispersants and/or foam formers. Suitable extenders are, for example, water, polar and unpolar organic chemical liquids, for example from the classes of the aromatic and nonaromatic hydrocarbons (such as paraffins, alkylbenzenes, alkylnaphthalenes, chlorobenzenes), of the alcohols and polyols (which can optionally also be substituted, etherified and/or esterified), of the ketones (such as acetone, cyclohexanone), esters (including fats and oils) and (poly)ethers, of the unsubstituted and substituted amines, amides, lactams (such as N-alkylpyrrolidones) and lactones, the sulphones and sulphoxides (such as dimethyl sulphoxide).

In the case of the use of water as an extender, organic solvents can, for example, also be used as cosolvents. Liquid solvents which are suitable are mainly aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, mineral oils and vegetable oils, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulphoxide, and water.

Solid carriers which are suitable are for example, ammonium salts and ground natural minerals, such as kaolins, clays, talc, chalk, quartz, attapulgite, montmorillonite or diatomaceous earth, and ground synthetic minerals, such as highly-disperse silica, alumina and silicates; suitable solid carriers for granules are: for example crushed and fractionated natural rocks such as calcite, marble, pumice, sepiolite and dolomite, and synthetic granules of inorganic and organic meals, and granules of organic material such as sawdust, coconut shells, maize cobs and tobacco stalks; suitable emulsifiers and/or foam formers are: for example non-ionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates as well as protein hydrolysates; suitable dispersants are: for example lignin-sulphite waste liquors and methylcellulose.

Any plant genus or species can be used with the delivery methods and agricultural compositions described herein, including, but not limited to, monocots and dicots. See, e.g., U.S. Pat. No. 8,080,647 (Pioneer Hi Bred). Examples of plant genuses and species include, but are not limited to, corn (Zea mays), Brassica spp. (e.g., B. napus, B. rapa, B. juncea), castor, palm, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp. such as lemon, lime, orange, tangelo, tangerine, etc.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), Arabidopsis thaliana, oats (Avena spp.), barley (Hordeum spp.), leguminous plants such as guar beans, locust bean, fenugreek, garden beans, cowpea, mungbean, fava bean, lentils, and chickpea, vegetables, ornamentals, grasses and conifers. Vegetables include tomatoes (Lycopersicon esculentum), lettuce (e.g., Lactuca sativa), green beans (Phaseolus vulgaris), lima beans (Phaseolus limensis), peas (Pisium spp., Lathyrus spp.), and Cucumis species such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers include pines, for example, loblolly pine (Pinus taeda), slash pine (Pinus elliotii), ponderosa pine (Pinus ponderosa), lodgepole pine (Pinus contorta), and Monterey pine (Pinus radiata), Douglas fir (Pseudotsuga menziesii); Western hemlock (Tsuga canadensis), Sitka spruce (Picea glauca), redwood (Sequoia sempervirens), true firs such as silver fir (Abies amabilis) and balsam fir (Abies balsamea), and cedars such as Western red cedar (Thuja plicata) and Alaska yellow cedar (Chamaecyparis nootkatensis).

The present invention is explained in greater detail in the following non-limiting Example.

### Example 1

In this Example, we tested the mobility of synthetic and virus-based model nanopesticides by combining soil column experiments with computational modelling. We found that Tobacco mild green mosaic virus and Cowpea mosaic virus penetrate soil to a depth of at least 30 cm, and could therefore deliver nematicides to the rhizosphere, whereas Physalis mosaic virus remains in the first 4 cm of soil and would be more useful for the delivery of herbicides. Our experiments confirm that plant viruses are superior to synthetic mesoporous silica nanoparticles and poly(lactic-co-glycolic acid) for the delivery and controlled release of pesticides can be used as pesticide delivery systems.

Most of the nanopesticides investigated thus far are based on synthetic or natural polymers, metallic compounds or liposomes, which tend to persist in the environment. As a biodegradable alternative, nanopesticides can be developed from plant viruses, either in the form of virus-like particles (VLPs), which do not carry nucleic acid and are therefore non-infectious, or as virus nanoparticles (VNPs), which include a virus genome and are potentially infectious. In the latter case, the EPA has already approved Tobacco mild green mosaic virus (TMGMV) as the herbicide Solvinix, which is produced by BioProdex for deployment against invasive tropical soda apple weed in the state of Florida.

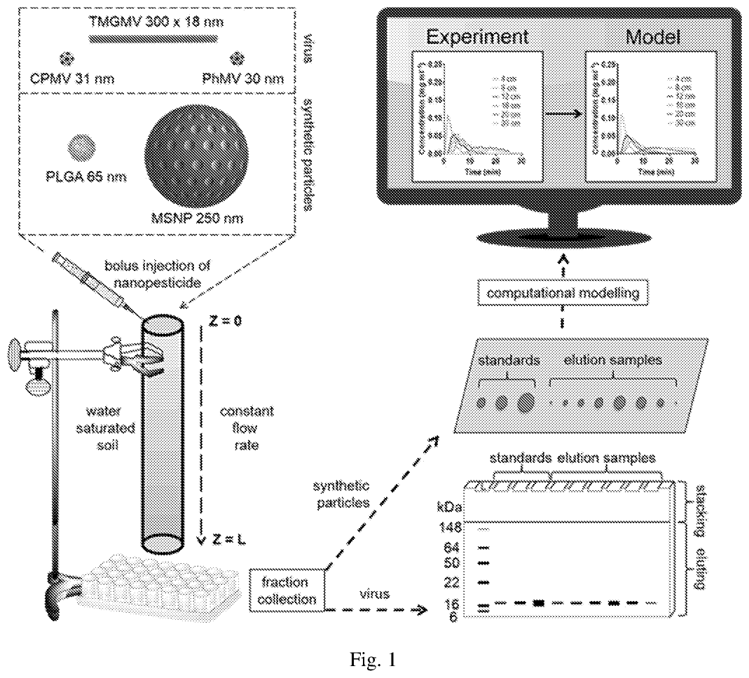

To investigate the potential of VLPs/VNPs as nanopesticides in more detail, we compared the behaviour of three viruses and two synthetic particle formulations in soil column experiments and computational models, as a way to gauge their ability to deliver pesticides to the rhizosphere and thus prevent infestation by root pests (FIG. 1). We tested two VNPs, based on the rod-like TMGMV and the icosahedral Cowpea mosaic virus (CPMV), and a VLP based on Physalis mosaic virus (PhMV). These were compared to mesoporous silica nanoparticles (MSNPs) and a poly(lactic-co-glycolic acid) formulation (PLGA), which have already been developed as synthetic nanopesticides.

### Materials and Methods

### Expression and Purification of Nanoparticles

TMGMV was obtained from Bioprodex, DegraFluorex Fluorescent PLGA nanoparticles were purchased from Phosphorex, and MSNPs functionalized with propylcarboxylic acids were obtained from Sigma-Aldrich. We resuspended 3 mg ml⁻¹ of PLGA and 1 mg ml⁻¹ of MSNP in distilled water and sonicated them using a Branson 2800 device (Cleanosonic) for 10 min to obtain homogeneous solutions. CPMV was propagated in Burpee black-eyed pea plants and purified as previously described. PhMV VLPs were prepared in ClearColi BL21 (DE3) cells.

### Bioconjugation of Cy5 to TMGMV Tyrosine Residues

TMGMV comprises 2,130 identical coat proteins arranged helically around a single-stranded RNA genome, forming a hollow rigid rod measuring 300×18 nm with a 4-nm internal channel. The external surface features two solvent-exposed tyrosine side chains (Tyr 2 and Tyr 139), which can be functionalized using diazonium coupling reactions. We used sulfo-Cy5-azide (Lumiprobe) to modify these Tyr residues. Briefly, we mixed 25 μl 0.68 M 3-ethynylaniline with 75 μl 3 M sodium nitrite (both Sigma-Aldrich) in 400 μl 0.3 M p-toluenesulfonic acid monohydrate (Thermo Fisher Scientific) for 1 h on ice. We then added 15 equivalents of the resulting diazonium salt (DS) to 2 mg ml⁻¹ TMGMV in 10 mM borate buffer (pH 8.8) for 30 min on ice. The particles were centrifuged at 112,000 g for 1 h on a 30% (w/v) sucrose cushion to separate the TMGMV-alkyne particles from the excess DS. TMGMV-alkyne was resuspended in 10 mM KP buffer (pH 7.4) overnight before adding sulfo-Cy5-azide via a Cu(I)-catalysed alkyne-azide cyclo-addition reaction. We added two equivalents of Cy5 per coat protein to 2 mg ml⁻¹ TMGMV-alkyne in the presence of 2 mM aminoguanidine, 2 mM L-ascorbic acid sodium salt and 1 mM copper(II) sulfate (all Sigma Aldrich) in 10 mM KP buffer (pH 7.4) on ice for 30 min. The particles were again centrifuged at 112,000 g for 1 hon a 30% (w/v) sucrose cushion to remove excess Cy5, and resuspended in 10 mM KP buffer (pH 7.4) overnight. Further purification to remove aggregates involved centrifugation at 16,000 g for 10 mM. TMGMV-Cy5 was eluted using PD Minitrap G-25 desalting columns (GE Healthcare) to remove free Cy5 dye.

### Bioconjugation of Cy5 to PhMV/CPMV Lysine Residues

CPMV comprises 180 identical coat proteins each displaying a surface-exposed lysine side chain. PhMV also comprises 180 identical coat proteins, but each displays four surface-exposed lysine side chains making 720 in total. CPMV and PhMV were labelled with sulfo-Cy5-NHS (Lumiprobe) using NHS-activated esters targeting the surface lysine residues. The reactions were carried out with a 1200-fold (CPMV) or 900-fold (PhMV) molar excess of sulfo-Cy5-NHS in 10 mM KP buffer (pH 7.0) at room temperature overnight, with agitation.

### Bioconjugation of Cy5 to MSNP Carboxylate Residues

Alkynes were conjugated to carboxylate groups on the MSNP surface using 1.5 mM propargylamine (Sigma-Aldrich) per gram of MSNP and 2.5 mM EDC in 10 mM HEPES buffer (pH 7.4). The reaction was allowed to proceed for 24 h at room temperature followed by an alkyne-azide click reaction induced by adding 250 nmoles of sulfo-Cy5-azide per gram of MSNP. The components were incubated at 4° C. with gentle agitation for 30 min using 1 mg ml⁻¹ MSNP in 10 mM KP buffer (pH 7.4) in the presence of 1 mM CuSO₄, 2 mM aminoguanidine and 2 mM ascorbate (all Thermo Fisher Scientific). MSNPs were purified by centrifugation at 7,000 g for 10 min and buffer exchange at least five times.

Encapsulation of Cy5 into TMGMV/CPMV/PhMV/MSNP Particles

Encapsulated formulations were prepared by mixing 1 mg ml⁻¹ of TMGMV, CPMV or PhMV with a 5000-fold molar excess of Cy5-Amine, or by mixing 250 nmoles of Cy5 per gram of MSNP in 10 mM KP buffer (pH 7.8) overnight at room temperature with agitation.

### UV/Vis Spectroscopy

The UV/vis spectra of native and modified TMGMV, CPMV, PhMV, PLGA and MSNP nanoparticles were determined using a NanoDrop Spectrophotometer (Thermo Fisher Scientific). The efficiency of Cy5 loading was determined based on the dye-to-carrier ratio and the Beer-Lambert law. TMGMV: ε(260 nm)=3 ml mg⁻¹ cm⁻¹, molecular weight of TMGMV=39.4×10⁶ g mol⁻¹. CPMV: ε(260 nm)=8.1 ml mg⁻¹ cm⁻¹, molecular weight of CPMV=5.6×10⁶ g mol⁻¹. Cy5: ε(647 nm)=271 000 M⁻¹ cm⁻¹, molecular weight of Cy5=747 g mol⁻¹. PLGA dye: ε(668 nm)=250 000 M⁻¹ cm⁻¹, molecular weight of PLGA dye=519 g mol⁻¹.

### Denaturing Gel Electrophoresis

We denatured 5 μg of native TMGMV, CPMV and PhMV at 100° C. for 5 min in the presence of 4×LDS loading dye (Thermo Fisher Scientific). Cy5-modified particles were denatured as described above using a loading dye lacking bromophenol blue. The samples were separated on 4-12% NuPage precast gels in 1×MOPS buffer (Thermo Fisher Scientific) for 40 min at 200 V and 120 mA, with SeeBlue Plus2 ladder size markers (Thermo Fisher Scientific). Gels were imaged before and after staining with Coomassie Brilliant Blue (0.25% w/v) using the FluorChem R imaging system under white light and MultiFluor red light.

### Agarose Gel Electrophoresis

We analyzed 3 μg of native CPMV, PhMV, PLGA and MSNP particles by 1.2% (w/v) agarose gel electrophoresis (1 h at 100 V) in 1×TBE running buffer in the presence of Nucleic Acid Gel Stain (GoldBio) diluted 1:20 000. Gels were imaged before and after staining with Coomassie Brilliant Blue (0.25% w/v) as above.

### Transmission Electron Microscopy

Formvar copper grids coated with carbon film (Electron Microscopy Sciences) were glow discharged to render the surface more hydrophilic using the PELCO easiGlow operating system. Drops of TMGMV, CPMV, PhMV or PLGA (10 μl, 1 mg ml⁻¹) were deposited onto the grids for 2 min at room temperature. The grids were then washed twice with deionized water for 30 s and subsequently stained twice with 2% (w/v) uranyl acetate for another 45 s. MSNP (10 μl, 1 mg ml⁻¹) was deposited onto grids and allowed to dry-cast overnight. A Tecnai F-30 transmission electron microscope was used to capture images of the samples at 300 kV.

### Dynamic Light Scattering

A DynaPro NanoStar instrument (Wyatt Technology) was used to measure the hydrodynamic radius of TMGMV, CPMV, PhMV, PLGA and MSNP nanoparticles. The reported hydrodynamic radii and standard derivations correspond to the average of 30 measurements, each consisting of 100 runs.

### Size Exclusion Chromatography

Native and modified TMGMV, CPMV, PhMV and PLGA samples (200 μl, 1 mg ml⁻¹) were passed through a Superose 6 Increase column on the AKTA Explorer chromatography system (GE Healthcare) at a flow rate 0.5 ml min⁻¹ in 10 mM KP buffer (pH 7.0). The absorbance at 260 nm and 280 nm was recorded for all particles, the absorbance at 647 nm was recorded to confirm Cy5 conjugation/encapsulation, and the absorbance at 668 nm was recorded to confirm dye encapsulation in the proprietary PLGA nanoparticles.

### Fluorescent-Dye Release Profiling

The release of Cy5 from TMGMV, PhMV, CPMV and MSNP, and dye release from PLGA, was evaluated using a dialysis-based assay. Slide-A-Lyzer MINI dialysis units (10,000 MWCO, Thermo Fisher Scientific) were loaded with 1 mg of particles in 10 mM KP buffer (pH 7.0) in triplicate. The particles were dialyzed against 10 mM KP buffer (pH 7.0) at room temperature for 96 h. At time t=0, 1, 3, 6, 18, 24, 48, 72 and 96 h, 10 μl was extracted from each dialysis units and the remaining dye entrapment was measured by UV/Vis spectroscopy (TMGMV, CPMV, PhMV and MSNP) or imaged under the FluorChem R imaging system (PLGA).

### Soil Mobility of TMGMV, CPMV, PhMV, PLGA, MSNP and Free Cy5

Garden Magic Top Soil was packed at a density of 0.32 g cm⁻³ into a cylindrical column (28 mm diameter, top height 30 cm) and saturated with deionized water to remove air pockets. We then applied 1 mg of each nanoparticle formulation in 300 μl 10 mM KP buffer (pH 7.0) to the centre of the soil surface at the top of the column We added 10 mM KP buffer (pH 7.0) at a constant flow rate of 1.5 ml min⁻¹ and collected the eluent in 500-μL fractions. Up to 150 fractions were collected in each trial (two trials for each formulation and depth) and stored at 4° C.

The elution fractions containing TMGMV, PhMV or CPMV were analysed by SDS-PAGE to determine the mass of nanoparticles recovered in each elution fraction. CPMV was analysed on 4-12% NuPage pre-cast gels in 1×MOPS buffer. TMGMV and PhMV were analysed on 4-12% NuPage polyacrylamide SDS gels cast according to the Surecast Handcast protocol (Invitrogen). We mixed 23 μl of each elution fraction with 7 μl 5×SDS loading buffer and separated the samples for 1 h at 200 V and 120 mA with SeeBlue Plus2 ladder size and three standards containing known amounts of nanoparticles (0.5, 1 and 2 μg) for comparison. The gels were then incubated in 20% (v/v) methanol and 10% (v/v) acetic acid in water 30 min before staining with Coomassie Brilliant Blue (0.25% w/v) for an additional 30 min. The gels were imaged using the AlphaImager HP system (Protein Simple) under white light and the FluorChem R system under MultiFluor red light. The elution fractions containing PLGA and MSNP were imaged as 20-μl droplets on Parafilm on the FluorChem R imaging system under MultiFluor red light in the presence of the nanoparticle standards described above.

All nanoparticles were imaged in triplicate and the images were analysed using ImageJ The area under the curve (AUC) of the standards was used to create a linear standard curve relating the AUC of the elution samples to the total mass of nanoparticles present in the corresponding elution fraction. Finally, fractions that appeared to contain no nanoparticles were centrifuged at 160,000 g for 3 h and the pellet was resuspended in 1 mL 10 mM KP buffer (pH 7.0) for SDS-PAGE analysis to determine the recovered mass of nanoparticles.

### Computational Methods

The nanoparticle mass density distribution in fluid (i.e., interstitial soil space) changes as a function of the column depth z and time t according to Eq. (1):

$$\frac{\partial {Ω}_{NP}}{\partial t} + \frac{Q}{A ε} \frac{\partial {Ω}_{NP}}{\partial z} = {D}_{NP} \frac{{\partial }^{2} {Ω}_{NP}}{\partial {z}^{2}} + ( \frac{1 - ε}{ε} ) ) ϕ {R}_{NPS} , 0 < z < L \qquad ( 1 )$$

where Ω_{NP} [mg cm⁻³] is the mass density of nanoparticles in the soil interstitial space at any location in the column, Q is the volume flow [cm³ min⁻¹], ε is the fluid fraction of volume (or cross-sectional area) in the column, A [cm²] is the cross-sectional area of the column, D_{NP} [cm² min⁻¹] is the dispersion constant of the nanoparticle, and ϕ[cm⁻¹] is the surface area to volume of soil. The irreversible rate of adsorption of nanoparticles onto the soil surface from the fluid is R_{NPS} [mg cm⁻² min⁻¹]. With and without pesticides, the nanoparticle adsorption process is assumed to be a first-order reaction as shown in Eq. (2):

R_{NPS}=−k_{NPS}Ω_{NP} (2)

The available soil surface, which changes negligibly, is incorporated into the rate constant k_{NPS} [cm min⁻¹]. As Ω_{NP} decreases with reaction, the nanoparticle attachment to soil increases as shown in Eq. (3):

$$\frac{\partial {Ω}_{NPS}}{\partial t} = - ϕ {R}_{NPS} \qquad ( 3 )$$

where Ω_{NPS} [mg cm⁻³] is the number density of nanoparticles bound to soil at any location in the column.

The pesticide mass concentration C_{P} [mg cm⁻³] distribution in fluid changes as shown in Eq. (4):

$$\frac{\partial {C}_{P}}{\partial t} + \frac{Q}{A ε} \frac{\partial {C}_{P}}{\partial z} = {D}_{P} \frac{{\partial }^{2} {C}_{P}}{\partial {z}^{2}} - {R}_{PF} + ( \frac{1 - ε}{ε} ) {ϕR}_{PS} , 0 < z < L \qquad ( 4 )$$

where D_{P} [cm² min⁻¹] is the dispersion constant of the pesticide. The irreversible release rate R_{PF} [mg cm⁻³ min⁻¹] of pesticide from nanoparticles in fluid is shown in Eq. (5):

R_{PF}=−k_{PF}C_{NPF} (5)

where C_{NPF} [mg cm⁻³] is the mass concentration of pesticide bound to nanoparticles in fluid at any location in the column and k_{PF} [min⁻¹] is the rate constant of pesticide dissociation from nanoparticles in fluid. The irreversible adsorption rate R_{PS} [mg cm⁻² min⁻¹] of free pesticide in fluid onto soil surface is shown in Eq. (6):

R_{PS}=−k_{PS}C_{P} (6)

where k_{PS} [cm min⁻¹] is the rate constant of pesticide absorption onto soil. The free pesticide mass concentration C_{PS} [mg cm⁻³] bound to soil changes according to Eq. (7):

$$\frac{\partial {C}_{P S}}{\partial t} = - ϕ ( {R}_{PS} + {R}_{P} ) \qquad ( 7 )$$

where R_{P} [min⁻¹] is the irreversible rate of pesticide ‘transfer’ from nanoparticles onto soil.

The concentration change of pesticide attached to nanoparticles in fluid is therefore shown in Eq. (8):

$$\frac{\partial {C}_{NPF}}{\partial t} + \frac{Q}{A ε} \frac{\partial {C}_{NPF}}{\partial z} = {D}_{NP} \frac{{\partial }^{2} {C}_{NPF}}{\partial {z}^{2}} + {R}_{PF} + ( \frac{1 - ε}{ε} ) ϕ {R}_{PNP} \qquad ( 8 )$$

where R_{PNP} [mg cm⁻² min⁻¹] is the irreversible adsorption rate of pesticide attached to nanoparticles onto the soil determined according to Eq. (9):

R_{PNP}=−k_{NPS}C_{NPF} (9)

where k_{NPS} [cm min⁻¹] is the rate constant of pesticide attached to nanoparticles that adsorb to soil. This rate process has the same rate constant as the rate process of nanoparticles adsorption onto soil (R_{NPS}), as defined in Eq. (10):

$$- {k}_{NPS} = \frac{{R}_{PNP}}{{C}_{NPF}} = \frac{{R}_{NPS}}{{Ω}_{NP}} \qquad ( 10 )$$

The pesticide concentration C_{NPS} [mg cm⁻³] in nanoparticles on soil changes according to Eq. (11):

$$\frac{\partial {C}_{NPS}}{\partial t} = ϕ ( {R}_{P} - {R}_{PNP} ) \qquad ( 11 )$$

The irreversible rate of pesticide transfer from nanoparticles to the soil with rate constant k_{P} [min⁻¹] is therefore shown in Eq. (12):

R_{P}=−k_{P}C_{NPS} (12)

Initially, there is no nanoparticle or pesticide in the soil space (0<z<L):

t=0: Ω_{NP}=0;Ω_{NPS}=0;C_{NPF}=0;C_{NPS}=0;C_{P}=0;C_{PS}=0;

At the input (top of the cylindrical column) z=0, a solution of volume V₀ with nanoparticle density Ω_{NP}⁰ and pesticide concentration C_{NPF}⁰ is injected. The total number of nanoparticles injected is N_{NP}⁰=V₀Ω_{NP}⁰. Fluid flows through the interstitial soil space at volume rate Q. The nanoparticles are transported into the column over a time interval 0 to t₁ according to Eq. (13):

$${N}_{NP}^{0} = {{\int }_{0}}^{{t}_{1}} Q {Ω}_{NP}^{0} dt = Q {Ω}_{NP}^{0} {t}_{1} ⟹ {t}_{1} = \frac{{N}_{NP}^{0}}{Q {Ω}_{NP}^{0}} \qquad ( 13 )$$

Therefore, at the entrance of the cylinder, mass flow rate balances must be specified for the nanoparticles and pesticide. For the nanoparticles, the input nanoparticle mass density is derived as shown in Eq. (14):

$$z = 0 : Q {Ω}_{NP} = \{ \begin{matrix}Q {Ω}_{NP}^{0} , & 0 < t \leq {t}_{1} \\ 0 , & t < {t}_{1}\end{matrix} ⟹ {Ω}_{NP} = \{ \begin{matrix}{Ω}_{NP}^{0} , & 0 < t \leq {t}_{1} \\ 0 , & t > {t}_{1}\end{matrix} \qquad ( 14 )$$

For the pesticide, which is carried by the nanoparticle, the input mass concentration is derived as shown in Eq. (15):

$$z = 0 : Q {Ω}_{NPF} = \{ \begin{matrix}{QC}_{NPF}^{0} , & 0 < t \leq {t}_{1} \\ 0 , & t > {t}_{1}\end{matrix} ⟹ {C}_{NP} = \{ \begin{matrix}{C}_{NPF}^{0} , & 0 < t \leq {t}_{1} \\ 0 , & t > {t}_{1}\end{matrix} \qquad ( 15 )$$

Because no dissolved pesticide enters, then z=0 and C_{P}=0. In the fluid leaving the column, the concentrations of nanoparticle, pesticide in nanoparticle, and free pesticide can be represented by X. The mass flow of X from inside the cylinder (L⁻) and to outside the cylinder (L⁺) is shown in Eq. (16):

$$z = L {: [ QX - A ε D \frac{\partial X}{\partial z} ]}_{L} = {[ QX ]}_{L} , X ∈ \{ {Ω}_{NP} , {C}_{NPF} , {C}_{P} \} \qquad ( 16 )$$

The volume flow rate, nanoparticle density and pesticide concentration are continuous across the output boundary of the cylinder, which implies that the gradients must vanish as shown in Eq. (17):

$$z = L : \frac{\partial {Ω}_{NP}}{\partial z} = 0 , \frac{\partial {C}_{P}}{\partial z} = 0 , \frac{\partial {C}_{CPF}}{\partial z} = 0 \qquad ( 17 )$$

For the simultaneous numerical solution of the partial differential equations, the equations were first transformed into their dimensionless form. These dimensionless equations were solved using partial differential equation solver function “pdepe” (MATLAB).

### Detailed Computational Methods for the Numerical Solution to the Model of Nanopesticide Transport Through the Soil

For the numerical solution of simultaneous partial differential equations, we used the MATLAB code “pdepe”. For numerical stability, we added a “false” diffusion term for equations involving the soil particles:

$$\frac{\partial {Ω}_{NPS}}{\partial t} = {D}_{F} \frac{{\partial }^{2} {Ω}_{NPS}}{\partial {z}^{2}} + ϕ {k}_{NPS} {Ω}_{NP}$$

$$\frac{\partial {C}_{NPS}}{\partial t} = {D}_{F} \frac{{\partial }^{2} {C}_{NPS}}{\partial {z}^{2}} + ϕ ( {R}_{P} - {R}_{PNP} )$$

$$\frac{\partial {C}_{PS}}{\partial t} = {D}_{F} \frac{{\partial }^{2} {C}_{PS}}{\partial {z}^{2}} - ϕ ( {R}_{P S} + {R}_{P} )$$

where the coefficient D_{F} [cm² min⁻¹] is given an arbitrarily small value. The boundary conditions for these modified equations are

$$z = 0 : {Ω}_{NPS} = 0 ; {C}_{NPS} = 0 ; {C}_{PS} = 0$$

$$z = L : \frac{\partial {Ω}_{NPS}}{\partial z} = 0 , \frac{\partial {C}_{NPS}}{\partial z} = 0 , \frac{\partial {C}_{PS}}{\partial z} = 0$$

### Dimensionless Forms

We can express the nanoparticle density as a function of fluid volume within the soil column in dimensionless form by defining the dimensionless variables:

$${t}^{′} = \frac{Q}{LA ε} t {z}^{′} = \frac{z}{L}$$

where Q is the constant volume flow of species through voids in soil, t is the time variable, L is the column length, and A is the constant cross-sectional area of the column.

Also, the dimensionless forms of pesticide concentration and nanoparticle density are

$${Ω}_{NP}^{′} = \frac{{Ω}_{NP}}{{Ω}_{NP}^{0}} ; {Ω}_{NPS}^{′} = \frac{{Ω}_{NPS}}{{Ω}_{NP}^{0}} ; {C}_{NPF}^{′} = \frac{{C}_{NPF}}{{C}_{NPF}^{0}} ; {C}_{NPS}^{′} = \frac{{C}_{NPS}}{{C}_{NPF}^{0}} ; {C}_{P}^{′} = \frac{{C}_{P}}{{C}_{NPF}^{0}} ; {C}_{PS}^{′} = \frac{{C}_{PS}}{{C}_{NPF}^{0}}$$

Starting with the equation for nanoparticle mass density in fluid:

$$\frac{\partial {Ω}_{NP}}{\partial t} + \frac{Q}{A ε} \frac{\partial {Ω}_{NP}}{\partial z} = {D}_{NP} \frac{{\partial }^{2} {Ω}_{NP}}{\partial {z}^{2}} + ( \frac{1 - ε}{ε} ) ϕ {R}_{NPS} , 0 < z < L ,$$

we substitute the dimensionless variables:

$$\frac{Q {Ω}_{N P}^{0}}{LA ε} \frac{\partial {Ω}_{NP}^{′}}{\partial {t}^{′}} + \frac{Q {Ω}_{N P}^{0}}{LA ε} \frac{\partial {Ω}_{NP}^{′}}{\partial {z}^{′}} = \frac{{Ω}_{NP}^{0} {D}_{NP}}{{L}^{2}} \frac{{\partial }^{2} {Ω}_{NP}^{′}}{\partial {z}^{′2}} - ( \frac{1 - ε}{ε} ) ϕ {Ω}_{NP}^{0} {k}_{NPS} {Ω}_{NP}^{′} , 0 < {z}^{′} < 1$$

Dividing by the coefficient of the first term yields

$$\frac{\partial {Ω}_{NP}^{′}}{\partial t} + \frac{\partial {Ω}_{NP}^{′}}{\partial z} = [ \frac{A ε {D}_{NP}}{QL} ] \frac{{\partial }^{2} {Ω}_{NP}^{′}}{\partial {z}^{′2}} - [ \frac{LA εϕ {k}_{NPS}}{Q} ] [ \frac{1 - ε}{ε} ] {Ω}_{NP}^{′} , 0 < {z}^{′} < 1 \qquad ( 1 )$$

Starting with the equation for mass density of nanoparticles attached to soil particles

$$\frac{\partial {Ω}_{NPS}}{\partial t} = {D}_{F} \frac{{\partial }^{2} {Ω}_{NPS}}{\partial {z}^{2}} + ϕ {k}_{NPS} {Ω}_{N P} , 0 < z < L ,$$

we substitute the dimensionless variables:

$$\frac{Q {Ω}_{NP}^{0}}{LA ε} \frac{\partial {Ω}_{N P S}^{′}}{\partial {t}^{′}} = \frac{{D}_{F} {Ω}_{NP}^{0}}{{L}^{2}} \frac{{\partial }^{2} {Ω}_{NPS}^{′}}{\partial {z}^{2}} + ϕ {k}_{NPS} {Ω}_{NP}^{0} {Ω}_{NP}^{′} , 0 < {z}^{′} < 1$$

Dividing by the coefficient of the first term yields

$$\frac{\partial {Ω}_{NPS}^{′}}{\partial {t}^{′}} = [ \frac{{D}_{F} A ε}{QL} ] \frac{{\partial }^{2} {Ω}_{NPS}^{′}}{\partial {z}^{′ 2}} + [ \frac{LA εϕ {k}_{NPS}}{Q} ] {Ω}_{NPS}^{′} , 0 < {z}^{′} < 1 \qquad ( 2 )$$

The equation for pesticide dissolved in fluid can be written as:

$$\frac{\partial {C}_{P}}{\partial t} + \frac{Q}{A ε} \frac{\partial {C}_{P}}{\partial z} = {D}_{P} \frac{{\partial }^{2} {C}_{P}}{\partial {z}^{2}} - {R}_{PF} + ( \frac{1 - ε}{ε} ) ϕ {R}_{PS} , 0 < z < L$$

$$\frac{{QC}_{NPF}^{0}}{LA ε} \frac{\partial {C}_{P}^{′}}{\partial {t}^{′}} + \frac{{QC}_{NPF}^{0}}{LA ε} \frac{\partial {C}_{P}^{′}}{\partial {z}^{′}} = \frac{{D}_{P} {C}_{NPF}^{0}}{{L}^{2}} \frac{{\partial }^{2} {C}_{P}^{′}}{\partial {z}^{′ 2}} + {k}_{PF} {C}_{NPF}^{0} {C}_{NPF}^{′} - ( \frac{1 - ε}{ε} ) ϕ [ {k}_{PS} {C}_{NPF}^{0} ] {C}_{P}^{′} 0 < {z}^{′} < 1$$

After the substitution of dimensionless variables, we find:

Dividing by the coefficient of the first term yields:

$$\frac{\partial {C}_{P}^{′}}{\partial {t}^{′}} + \frac{\partial {C}_{P}^{′}}{\partial {z}^{′}} = [ \frac{A ε {D}_{P}}{QL} ] \frac{{\partial }^{2} {C}_{P}^{′}}{\partial {z}^{′ 2}} + [ \frac{LA ε {k}_{PF}}{Q} ] {C}_{NPF}^{′} - [ \frac{LA εϕ {k}_{PS}}{Q} ] [ \frac{1 - ε}{ε} ] {C}_{P}^{′} , 0 < {z}^{′} < 1 \qquad ( 3 )$$

The equation for pesticide attached to nanoparticles in fluid can be written as:

$$\frac{\partial {C}_{NPF}}{\partial t} + \frac{Q}{A ε} \frac{\partial {C}_{NPF}}{\partial z} = {D}_{NP} \frac{{\partial }^{2} {C}_{NPF}}{\partial {z}^{2}} + {R}_{PF} + ( \frac{1 - ε}{ε} ) ϕ {R}_{PNP} , 0 < z < L$$

After the substitution of dimensionless variables, we find:

$$\frac{{QC}_{NPF}^{0}}{LA ε} \frac{\partial {C}_{NPF}^{′}}{\partial {t}^{′}} + \frac{{QC}_{NPF}^{0}}{LA ε} \frac{\partial {C}_{NPF}^{′}}{\partial {z}^{′}} = \frac{{D}_{NP} {C}_{NPF}^{0}}{{L}^{2}} \frac{{\partial }^{2} {C}_{NPF}^{′}}{\partial {z}^{′ 2}} - {k}_{PF} {C}_{NPF}^{0} {C}_{NPF}^{′} - ( \frac{1 - ε}{ε} ) ϕ {k}_{NPS} {C}_{NPF}^{0} {C}_{NPF}^{′}$$

Dividing by the coefficient of the first term yields:

$$\frac{\partial {C}_{NPF}^{′}}{\partial {t}^{′}} + \frac{\partial {C}_{NPF}^{′}}{\partial {z}^{′}} = [ \frac{A ε {D}_{NP}}{QL} ] \frac{{\partial }^{2} {C}_{NPF}^{′}}{\partial {z}^{′ 2}} - [ \frac{LA ε {k}_{PF}}{Q} ] {C}_{NPF}^{′} + [ \frac{LA εϕ {k}_{NPS}}{Q} ] ( \frac{1 - ε}{ε} ) {C}_{NPF}^{′} , 0 < {z}^{′} < 1 \qquad ( 4 )$$

The equation for pesticide attached to nanoparticles on soil particles:

$$\frac{\partial {C}_{NPS}}{\partial t} = {D}_{F} \frac{{\partial }^{2} {C}_{NPS}}{\partial {z}^{2}} + ϕ ( {R}_{P} - {R}_{PNP} )$$

After the substitution of dimensionless variables, we find:

$$\frac{{QC}_{NPF}^{0}}{LA ε} \frac{\partial {C}_{NPS}^{′}}{\partial {t}^{′}} = \frac{{D}_{F} {C}_{NPF}^{0}}{{L}^{2}} \frac{{\partial }^{2} {C}_{NPS}^{′}}{\partial {z}^{′ 2}} - ϕ ( {k}_{P} {C}_{NPF}^{0} {C}_{NPS}^{′} - {k}_{NPS} {C}_{NPF}^{0} {C}_{NPF}^{′} )$$

Dividing by the coefficient of the first term yields:

$$\frac{\partial {C}_{NPS}^{′}}{\partial {t}^{′}} = [ \frac{{D}_{F} A ε}{QL} ] \frac{{\partial }^{2} {C}_{NPS}^{′}}{\partial {z}^{′ 2}} - [ \frac{LA εϕ {k}_{P}}{Q} ] {C}_{NPS}^{′} + [ \frac{LA εϕ {k}_{NPS}}{Q} ] {C}_{NPF}^{′} \qquad ( 5 )$$

The equation for pesticide adsorbed by soil particles can be written as:

$$\frac{\partial {C}_{PS}}{\partial t} = {D}_{F} \frac{{\partial }^{2} {C}_{PS}}{\partial {z}^{2}} - ϕ ( {R}_{PS} + {R}_{P} )$$

After the substitution of dimensionless variables, we find:

$$\frac{{QC}_{NPF}^{0}}{LA ε} \frac{\partial {C}_{PS}^{′}}{\partial {t}^{′}} = \frac{{D}_{F} {C}_{NPF}^{0}}{{L}^{2}} \frac{{\partial }^{2} {C}_{PS}^{′}}{\partial {z}^{2}} + ϕ ( {k}_{PS} {C}_{NPF}^{0} {C}_{P}^{′} + {k}_{P} {C}_{NPF}^{0} {C}_{NPS}^{′} )$$

Dividing by the coefficient of the first term yields:

$$\frac{\partial {C}_{PS}^{′}}{\partial {t}^{′}} = [ \frac{{D}_{F} A ε}{QL} ] \frac{{\partial }^{2} {C}_{PS}^{′}}{\partial {z}^{2}} + [ \frac{LA εϕ {k}_{PS}}{Q} ] {C}_{P}^{′} + [ \frac{LA εϕ {k}_{P}}{Q} ] {C}_{NPS}^{′} \qquad ( 6 )$$

Equations 1-6 can be written as:

$$\frac{\partial {Ω}_{NP}^{′}}{\partial {t}^{′}} + \frac{\partial {Ω}_{NP}^{′}}{\partial {z}^{′}} = \frac{1}{{Pe}_{NP}} \frac{{\partial }^{2} {Ω}_{NP}^{′}}{\partial {z}^{′ 2}} - {Da}_{NP} [ \frac{1 - ε}{ε} ] {Ω}_{NP}^{′} \qquad ( 1 )$$

$$\frac{\partial {Ω}_{NPS}^{′}}{\partial {t}^{′}} = \frac{1}{{Pe}_{NPS}} \frac{{\partial }^{2} {Ω}_{NPS}^{′}}{\partial {z}^{′ 2}} + {Da}_{NP} {Ω}_{NP}^{′} \qquad ( 2 )$$

$$\frac{\partial {C}_{P}^{′}}{\partial {t}^{′}} + \frac{\partial {C}_{P}^{′}}{\partial {z}^{′}} = \frac{1}{{Pe}_{D}} \frac{{\partial }^{2} {C}_{P}^{′}}{\partial {z}^{′ 2}} + {Da}_{PF} {C}_{NPF}^{′} - {Da}_{PS} [ \frac{1 - ε}{ε} ] {C}_{P}^{′} \qquad ( 3 )$$

$$\frac{\partial {C}_{NPF}^{′}}{\partial {t}^{′}} + \frac{\partial {C}_{NPF}^{′}}{\partial {z}^{′}} = \frac{1}{{Pe}_{NP}} \frac{{\partial }^{2} {C}_{NPF}^{′}}{\partial {z}^{′ 2}} - {Da}_{PF} {C}_{NPF}^{′} + {Da}_{NP} ( \frac{1 - ε}{ε} ) {C}_{NPF}^{′} \qquad ( 4 )$$

$$\frac{\partial {C}_{NPS}^{′}}{\partial {t}^{′}} = \frac{1}{{Pe}_{NPS}} \frac{{\partial }^{2} {C}_{NPS}^{′}}{\partial {z}^{′ 2}} - {Da}_{P} {C}_{NPS}^{′} + {Da}_{NP} {C}_{NPF}^{′} \qquad ( 5 )$$

$$\frac{\partial {C}_{PS}^{′}}{\partial {t}^{′}} = \frac{1}{{Pe}_{NPS}} \frac{{\partial }^{2} {C}_{PS}^{′}}{\partial {z}^{2}} + {Da}_{PS} {C}_{P}^{′} + {Da}_{P} {C}_{NPS}^{′} \qquad ( 6 )$$

### Results

### Nanopesticide Characterization

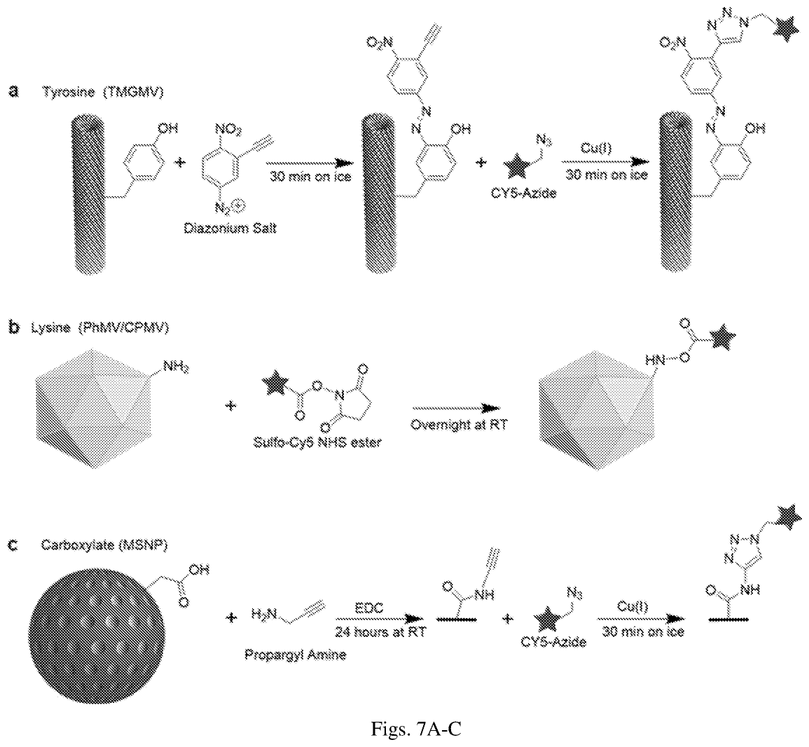

The fluorophore Cyanine 5 (Cy5) has similar physicochemical properties to conventional pesticides but is easier to detect, so we used it as a model compound. Cy5 was either conjugated to the external surface of, or passively encapsulated within, TMGMV, CPMV, PhMV and MSNP particles (FIG. 7). Degradex PLGA nanoparticles encapsulating a red fluorophore with spectral properties similar to Cy5 were obtained from Phosphorex. TMGMV, CPMV, PhMV, MSNP and PLGA formulations with and without dyes were characterized by a combination of transmission electron microscopy (TEM), dynamic light scattering (DLS), UV/Vis spectroscopy, size exclusion chromatography (SEC) and denaturing gel electrophoresis (SDS-PAGE) or agarose gel electrophoresis, to confirm particle integrity and dye loading efficiency. The capacity of TMGMV was 9.9 nmol mg⁻¹ or 390 dye molecules per TMGMV-Cy5 particle (denotes the conjugated version) but only 5.3 nmol mg⁻¹ or 210 dye molecules per TMGMV*Cy5 particle (denotes the encapsulated version). For PhMV, the corresponding loads were 12.7 nmol mg⁻¹ or 60 dye molecules per PhMV-Cy5 and 11.7 nmol mg⁻¹ or 55 dye molecules per PhMV*Cy5. For CPMV, the corresponding loads were 6.2 nmol mg⁻¹ or 35 dye molecules per CPMV-Cy5 and 2.3 nmol mg⁻¹ or 15 dye molecules per CPMV*Cy5. The synthetic MSNP formulation was similar in capacity to CPMV (6.4 nmol mg⁻¹ for MSNP-Cy5 and 4.3 nmol mg⁻¹ for MSNP*Cy5) whereas the PGLA formulation had the lowest capacity (1.2 nmol mg⁻¹ for PLGA*Dye).

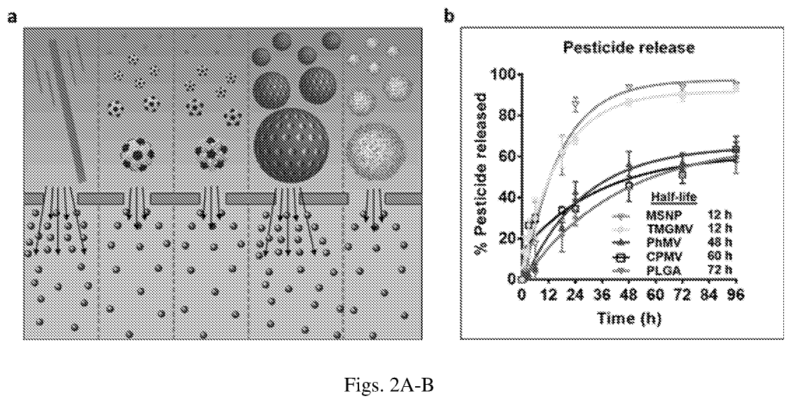

The release profile of passively encapsulated Cy5 (FIG. 2) was determined by dialysing 1 ml of a 1 mg ml⁻¹ solution of TMGMV*Cy5, CPMV*Cy5, PhMV*Cy5, MSNP*Cy5 or PLGA*Dye against 10 mM potassium phosphate (KP) buffer (pH 7.0) for 96 h at room temperature. At time t=0, 1, 3, 6, 18, 24, 48, 72 and 96 h, 10 μl was extracted from each dialysis and the remaining entrapped dye was measured by UV/Vis spectroscopy (TMGMV, CPMV, PhMV and MSNP) or imaged using the FluorChem R imaging system (Protein Simple) under MultiFluor red light (PLGA). The approximate half-life, defined as the time necessary for 50% of the fluorophore to be released from its carrier, was calculated for TMGMV (t_{1/2}=12 h), CPMV (t_{1/2}=60 h), PhMV (t_{1/2}=48 h), MSNP (t_{1/2}=12 h) and PLGA (t_{1/2}=72 h). Two distinct release profiles were observed, reflecting the Cy5 entrapment methodology used in each formulation. For TMGMV and MSNP, Cy5 is not entrapped in a confined structure because the internal channel of TMGMV is uncapped at both ends and the mesopores of MSNP are similarly open to the surrounding medium, potentially explaining the faster release rate. For CPMV and PhMV, Cy5 is encapsulated within the protein shell and the PGLA nanoparticle encapsulates the dye in its hydrophobic core, hence the slower release. The observed release profiles may not precisely replicate pesticide release in a real soil environment, which is rich in various minerals and organic matter that might interact with either the carriers or the pesticide molecules, as discussed below.

### Soil Transport Behavior

To establish the soil transport behaviour of each formulation, we conducted mobility studies using a cylindrical column of diameter 28 mm packed with topsoil at a constant bulk density of 0.32 g cm⁻³. This was the maximum density achievable under our experimental conditions, but the density of soil in real environments can be higher (0.6-1.6 g cm⁻³) due to compaction effects with depth and over time. We injected a bolus containing 1 mg of each formulation with and without conjugated or infused dye molecules at the top of the soil column and saturated the column with water at a constant flow rate of 1.5 cm³ min⁻¹. The eluent was collected at the base of the column in 500-μl fractions. Up to 200 fractions were collected in each trial (two trials per depth for each formulation). Fractions containing virus-based formulations were analysed by SDS-PAGE to determine the mass of recovered nanoparticles. The MSNP and PLGA elution fractions were imaged as 20-μ1 droplets on the FluorChem R imaging system under MultiFluor red light.

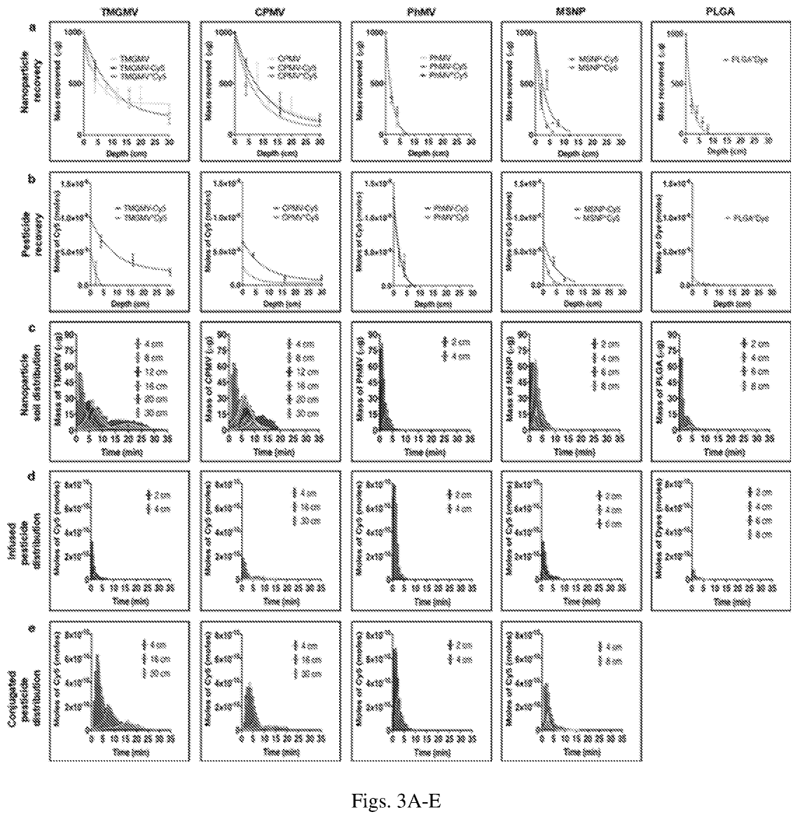

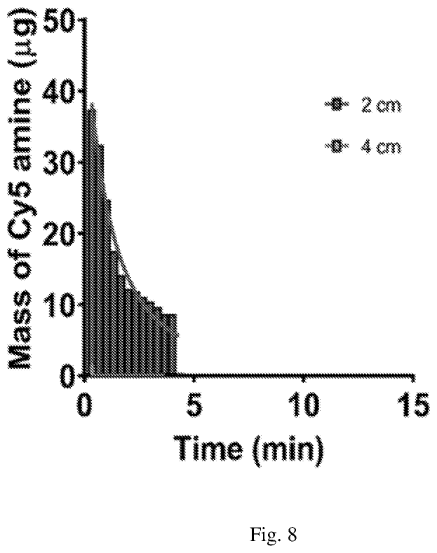

As a reference, we ran 500 μg of free Cy5 through a soil column with a smaller diameter of 10 mm Cy5 was unable to penetrate further than 4 cm through the soil because it bound strongly to the soil particles (FIG. 8). About 40% of the mass of injected Cy5 was recovered from a column with a soil depth of 2 cm. No matter which nanoparticle type was used as a carrier, the mobility of Cy5 within the column was significantly enhanced (FIG. 3). The best-performing carrier was TMGMV, which penetrated to a soil depth of 30 cm regardless of whether the cargo was conjugated or encapsulated (FIG. 3A). The spatiotemporal distribution of the nanoparticles and the Cy5 cargo was very similar, indicating that in each formulation the carrier and cargo were co-eluted (FIG. 3C-E). The quantity of encapsulated Cy5 that co-eluted with its carrier decreased with soil depth, indicating that a portion of the cargo was released over time.

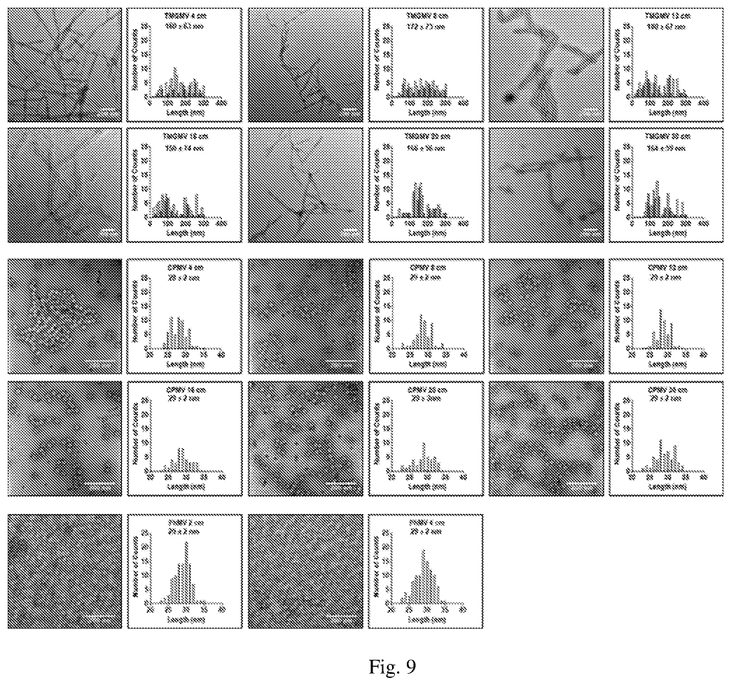

To determine the quantity of particles recovered from the soil transport experiment, we pooled all elution samples that showed no evidence of nanoparticles in SDS-PAGE analysis and centrifuged them at 112,000 g for 3 h to collect any trace amounts of the virus. The concentrated solutions were analysed by SDS-PAGE to calculate the residual mass of experimentally lost nanoparticles, and we found that it accounted for only ˜2.5% of the overall mass of particles injected (Table 1). TEM imaging of the eluted particles revealed that they remained intact (FIG. 9).

Elution samples lacking viral mass according to SDS-PAGE analysis were pooled and centrifuged at 112,000 g for 3 h to collect any trace amounts of virus. The concentrated solutions were analysed by SDS-PAGE gels to determine the residual mass of experimentally lost nanoparticles.

TABLE 1
tables of virus recovery from the empty elution samples

| | |
|---|---|
| Average Loss (μg) | % loss |

TMGMV

| | | | |
|---|---|---|---|
| 4 | cm | 16 | 1.6 |
| 8 | cm | 10 | 1 |
| 12 | cm | 44 | 4.4 |
| 16 | cm | 37 | 3.7 |
| 20 | cm | 26 | 2.6 |
| 30 | cm | 28 | 2.8 |

CMPV

| | | | |
|---|---|---|---|
| 4 | cm | 25 | 2.5 |
| 8 | cm | 22 | 12.2 |
| 12 | cm | 43 | 4.3 |
| 16 | cm | 32 | 3.2 |
| 20 | cm | 26 | 2.6 |
| 30 | cm | 40 | 4 |

PhMV

| | | | |
|---|---|---|---|
| 2 | cm | 0 | 0 |
| 4 | cm | 0 | 0 |
| 6 | cm | 0 | 0 |
| 8 | cm | 0 | 0 |

TMGMV-Cy5

| | | | |
|---|---|---|---|
| 4 | cm | 16 | 2.4 |
| 16 | cm | 37 | 6 |
| 30 | cm | 28 | 8.3 |

CPMV-Cy5

| | | | |
|---|---|---|---|
| 4 | cm | 26 | 2.6 |
| 16 | cm | 31 | 3.1 |
| 30 | cm | 15 | 1.5 |

PhMv-Cy5

| | | | |
|---|---|---|---|
| 2 | cm | 19 | 1.9 |
| 4 | cm | 35 | 3.5 |

TMGMV-Cy5-Amine

| | | | |
|---|---|---|---|
| 2 | cm | 35 | 3.5 |
| 4 | cm | 20 | 3 |

CPMV-Cy5-Amine

| | | | |
|---|---|---|---|
| 4 | cm | 24 | 2.4 |
| 16 | cm | 16 | 1.6 |
| 30 | cm | 19 | 1.9 |

PhMV-Cy5-Amine

| | | | |
|---|---|---|---|
| 2 | cm | 1.2 | 0.12 |
| 4 | cm | 1.8 | 0.18 |

In terms of soil transport behaviors, TMGMV and CPMV were able to penetrate through 30 cm of soil, whereas PhMV, MSNP and PLGA only penetrated 4 12 and 8 cm of soil, respectively. The mobility of the carriers in soil can therefore be ranked from highest to lowest as follows: TMGMV>>CPMV>>>MSNP>PLGA>PhMV. These data suggest that the PhMV, MSNP and PLGA formulations are not suitable for pesticide delivery deep into the soil, to target the rhizosphere, but may be suitable for the delivery of pesticides that must remain close to the surface, such as herbicides. In the latter context, PhMV demonstrated the greatest pesticide delivery capability within the first 4 cm of soil (FIG. 3B). The particle size may influence the mobility of the carriers, but there was no particular trend within the size range we tested. For example, the 250-nm MSNP particles penetrated further than the 65-nm PLGA formulation, which in turn penetrated further than the 30-nm PhMV particles, but the 31-nm CPMV particles were much more mobile than all of the above. This is interesting given that CPMV and PhMV are similar in size and geometry, so the remarkable difference in mobility must reflect their surface chemistries. Both CPMV and PhMV are proteinaceous, but the distinct amino acid sequences of their coat proteins ensure that CPMV carries a negative surface charge whereas PhMV is positive. Furthermore, the rod-like (300×18 nm) TMGMV particles were the most mobile of all, suggesting that the elongated shape may facilitate their transport through the soil. A high aspect ratio therefore appears to be a generally favorable property that facilitates movement between obstacles by influencing particle behavior in flowing liquids.

The concentration of Cy5 as a function of soil depth was higher when the dye was conjugated to the particles rather than encapsulated (FIG. 3B). This reflects the slower release of the conjugated dye from the carrier, allowing it to be carried further, whereas the encapsulated dye leaks more readily from the carrier and once released is rendered less mobile by its affinity for soil particles. Interestingly, Cy5 was released particularly rapidly from the TMGMV*Cy5 and MSNP*Cy5 formulations (4 and 6 cm penetration, respectively) which suggests that the electrostatic forces between Cy5 and the carboxylate residues of (i) the TMGMV interior and (ii) the MSNP mesopores are not strong enough to overcome the attraction between Cy5 and the soil. These results agree with the rapid loss of Cy5 observed in the dialysis assay (FIG. 2). Therefore, for field applications, the conjugated formulation appears superior to the encapsulated formulation.

Both TMGMV and CPMV were able to deliver Cy5 deep in the soil, but TMGMV-Cy5 showed by far the better performance We have previously shown that nematodes ingest nematicide-loaded TMGMV particles, which resulted in the death of 60% of the nematode population in liquid cultures within 24 h. To increase the efficacy, future TMGMV formulations can include cleavable linkers to promote the slow and controlled release of the pesticide at the root level, by making the linker pH sensitive. But in order to translate such pesticide formulations from the bench to the field, it is first necessary to establish the dose required to eradicate rhizosphere-dwelling pests. We therefore developed a mathematical model and validated it using our experimental data as discussed below.

### Computational Modelling of Pesticide Delivery

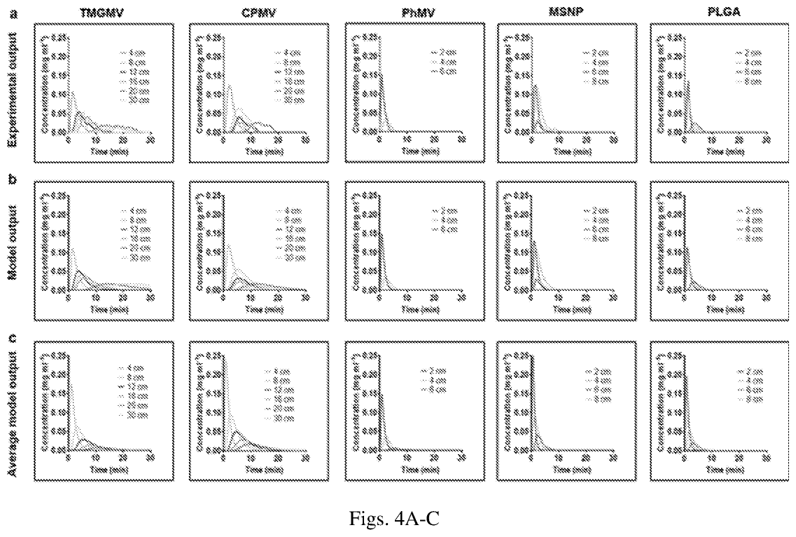

A model column of length L [cm] and constant cross-sectional area A [cm²] was filled with a mixture of stationary soil particles and fluid (FIG. 1). The input to this model was a known mass nanoparticles, with or without pesticide, introduced over a short period of time to the soil surface. The outputs were the concentrations of the nanoparticle Ω_{NP} [mg cm⁻³], the nanoparticle-pesticide formulation C_{NPS} [mg cm⁻³], and free pesticide C_{P} [mg cm⁻³] at the base of the soil column as a function of time for a specific depth of soil. Following the injection, fluid flow was established at top the column at a rate Q [cm³ min⁻¹]. Nanoparticles were subsequently transported through the void volume fraction □ [dimensionless] of the saturated soil column, with an adsorption surface per soil particle volume □ [cm⁻¹]. The soil particle density within the column was assumed to be uniform. The rates of nanoparticle degradation and pesticide deactivation were assumed to be negligible during the experiment, as confirmed empirically (FIG. 9). Nanoparticle binding to soil particles was modelled as a first-order irreversible reaction with rate constant k_{NPS} [cm min⁻¹] dependent on the nanoparticle size, aspect ratio and surface chemistry. The pesticide release rate was modelled as a first-order irreversible reaction with rate constant k_{PF} [min⁻¹]. The resulting free pesticide may move by convection and diffusion through the interstitial spaces, or bind to soil particles through a first-order irreversible reaction with rate constant k_{PS} [cm min⁻¹]. Although most constants could be defined from our experimental data, the system contained five unknowns: the dispersion constants of the nanoparticle D_{NP} and pesticide D_{P}, and the rate constants of nanoparticle absorption to soil k_{NPS}, pesticide absorption to soil k_{PS}, and pesticide release from nanoparticles in fluid k_{PF} These values were obtained by comparing the model output to the empirical data and minimizing the error in MATLAB. The optimal estimated values of the unknown model parameters (Table 2) were substituted into the model equations to obtain the predicted time distribution of the nanoparticle concentration for a specific depth of soil (FIG. 4). The resulting model outputs closely matched the empirical data, although the values of D_{NP} and k_{NPS} differed slightly for each depth due to experimental error caused by the need to use a new soil column in each test. Although the bulk density of the soil was kept constant across all experiments, the soil particle distribution and the soil packing may have differed from column to column. To compensate for these variables, the average values of D_{NP} and k_{NPS} at different depths were computed to model the average nanoparticle soil transport profile (FIG. 4C). The nanoparticle dispersion D_{NP} and rate of absorption to soil k_{NPS} determine the ability of a nanoparticle to carry pesticide deep in the soil. With greater mechanical dispersion, the nanoparticles become more widely distributed at a given soil depth over time. Therefore, mechanical dispersion greatly influences the concentration of nanoparticles at any given soil depth and time. The average D_{NP} of each nanoparticle can be ranked from highest to lowest as follows: TMGMV>CPMV>MSNP>PhMV>PLGA. As the absorption to the soil becomes stronger, the nanoparticles become less mobile. The average rate constant of nanoparticle absorption to soil k_{NPS} can also be ranked from highest to lowest as follows: MSNP>>>PLGA≈PhMV>>TMGMV>CPMV. The absolute errors between the empirical data and the model output varied between 10⁻³ and 10⁻⁹, which is excellent agreement (Table 2). The model confirms the superior mobility of TMGMV and its suitability to deliver pesticides to the rhizosphere.

TABLE 2
Optimized parameters.

| | | | TMGMV | CPMV | PhMV | MSNP | PLGA |
|---|---|---|---|---|---|---|---|
| 2 | cm | k_{NPS} | | | 9.69 × 10⁻⁰⁵ | 7.00 × 10⁻² | 9.81 × 10⁻⁰⁵ |
| | | D_{NP} | | | 0.72 | 0.50 | 0.51 |
| 4 | cm | k_{NPS} | 4.53 × 10⁻⁰⁵ | 1.53 × 10⁻⁰⁵ | 1.07 × 10⁻⁴ | 3.95 × 10⁻² | 1.22 × 10⁻⁴ |
| | | D_{NP} | 1.55 | 1.35 | 0.75 | 1.43 | 1.60 |
| 6 | cm | k_{NPS} | | | | 2.13 × 10⁻¹ | 8.92 × 10⁻⁵ |
| | | D_{NP} | | | | 2.07 | 0.98 |
| 8 | cm | k_{NPS} | 2.81 × 10⁻⁰⁵ | 1.88 × 10⁻⁰⁵ | | 1.7 × 10⁻¹ | 1.06 × 10⁻⁴ |
| | | D_{NP} | 1.34 | 1.44 | | 2.9758 | 1.08 |
| 12 | cm | k_{NPS} | 5.36 × 10⁻⁰⁵ | 3.89 × 10⁻⁰⁶ | | | |
| | | D_{NP} | 5.08 | 2.71 | | | |
| 16 | cm | k_{NPS} | 3.92 × 10⁻⁰⁵ | 5.11 × 10⁻⁰⁵ | | | |
| | | D_{NP} | 4.91 | 4.42 | | | |
| 20 | cm | k_{NPS} | 2.07 × 10⁻⁰⁵ | 2.58 × 10⁻⁰⁵ | | | |
| | | D_{NP} | 2.22 | 2.95 | | | |
| 30 | cm | k_{NPS} | 4.26 × 10⁻⁰⁵ | 5.39 × 10⁻⁰⁵ | | | |
| | | D_{NP} | 6.39 | 10.17 | | | |

| Average | k_{NPS} | 3.82 × 10⁻⁰⁵ | 2.3 × 10⁻⁰⁵ | 1.02 × 10⁻⁰⁴ | 1.236 × 10⁻⁰¹ | 1.04 × 10⁻⁰⁴ |
|---|---|---|---|---|---|---|
| | D_{NP} | 3.58 | 2.57 | 0.73 | 1.75 | 1.04 |
| STD | k_{NPS} | 1.19 × 10⁻⁰⁵ | 1.76 × 10⁻⁰⁵ | 6.93 × 10⁻⁰⁶ | 8.12 × 10⁻⁰² | 1.4 × 10⁻⁰⁵ |
| | D_{NP} | 2.14 | 1.26 | 0.02 | 1.04 | 0.45 |

| | | Cy5 |
|---|---|---|
| 2 cm | K_{PS} | 1.37 × 10⁻⁰⁴ |
| | Dₚ | 1.73 |

| | | | TMGMV*Cy5 | CPMV*Cy5 | PhMV*Cy5 | MSNP*Cy5 |
|---|---|---|---|---|---|---|
| 2 | cm | K_{PF} | | | 5.18 × 10⁻⁰³ | 5.08 × 10⁻⁰⁵ |
| 4 | cm | K_{PF} | 5.89 × 10⁻⁰⁴ | 2.27 × 10⁻⁰⁴ | 1.26 × 10⁻⁰³ | 3.75 × 10⁻⁰⁴ |
| 6 | cm | K_{PF} | | | | 1.18 × 10⁻⁰⁴ |
| 16 | cm | K_{PF} | | 5.46 × 10⁻⁰⁴ | | |
| 30 | cm | K_{PF} | | 3.49 × 10⁻⁰⁴ | | |

| | | | | | |
|---|---|---|---|---|---|
| Average | K_{PF} | 5.89 × 10⁻⁰⁴ | 1.05 × 10⁻⁰³ | 3.22 × 10⁻⁰³ | 2.03 × 10⁻⁰⁴ |
| STD | K_{PF} | NA | 8.63 × 10⁻⁰⁴ | 1.96 × 10⁻⁰³ | 1.33 × 10⁻⁰⁴ |

k_{NPS} [cm min⁻¹]: rate constant of nanoparticle absorption to soil.
D_{NP} [cm² min⁻¹]: dispersion constant of nanoparticles in the interstitial space.
k_{PS} [cm min⁻¹]: rate constant of pesticide absorption to soil.
D_{P} [cm² min⁻¹]: dispersion constant of pesticide in the interstitial space.
k_{PF} [min⁻¹]: rate constant of pesticide release from nanoparticles.

To quantify how well the computational outputs matched the empirical data, we calculated the difference (error) as follows:

$$\frac{\partial {Ω}_{NP}}{\partial t} + \frac{Q}{A ε} \frac{\partial {Ω}_{NP}}{\partial z} = {D}_{NP} \frac{{\partial }^{2} {Ω}_{NP}}{\partial {z}^{2}} + ( \frac{1 - ε}{ε} ) ϕ {R}_{NPS} , 0 < z < L$$

Error(z)=SUM(OmegaNPF_x(t,z)−OmegaNPF_x(t,z))\hat{ }2);

| | | TMGMV | CPMV | PhMV | MSNP | PLGA |
|---|---|---|---|---|---|---|
| 2 | cm | | | 3.07 × 10⁻³ | 1.31 × 10⁻³ | 1.67 × 10⁻³ |
| 4 | cm | 7.76 × 10⁻⁴ | 1.03 × 10⁻³ | 7.7 × 10⁻⁵ | 7.48 × 10⁻⁴ | 2.98 × 10⁻⁴ |
| 6 | cm | | | | 7.58 × 10⁻⁵ | 8.56 × 10⁻⁵ |
| 8 | cm | 1.98 × 10⁻⁴ | 2.06 × 10⁻³ | | 1.91 × 10⁻⁴ | 9.3 × 10⁻⁶ |
| 12 | cm | 9.96 × 10⁻⁵ | 6.71 × 10⁻⁴ | | | |
| 16 | cm | 7.48 × 10⁻⁴ | 3.07 × 10⁻⁴ | | | |
| 20 | cm | 7.35 × 10⁻⁴ | 1.86 × 10⁻³ | | | |
| 30 | cm | 2.44 × 10⁻³ | 4.36 × 10⁻⁴ | | | |

Error(z)=SUM(OmegaNPF_x(t,z)−OmegaNPF_x(t,z))\hat{ }2);

| | Cy5 |
|---|---|
| 2 cm | 7.2 × 10⁻⁰⁵ |

Error(z)=SUM(OmegaNPF_x(t,z)−OmegaNPF_x(t,z))\hat{ }2);

| | | TMGMV*Cy5 | CPMV*Cy5 | PhMV*Cy5 | MSNP*Cy5 |
|---|---|---|---|---|---|
| 2 | cm | | | 6.56 × 10⁻⁸ | 2.27 × 10⁻³ |
| 4 | cm | 6.59 × 10⁻⁵ | 3.66 × 10⁻⁸ | 1.24 × 10⁻⁸ | 6.66 × 10⁻⁴ |
| 6 | cm | | | | 1.65 × 10⁻⁴ |
| 16 | cm | | 7.82 × 10⁻⁹ | | |
| 30 | cm | | 1.71 × 10⁻⁸ | | |

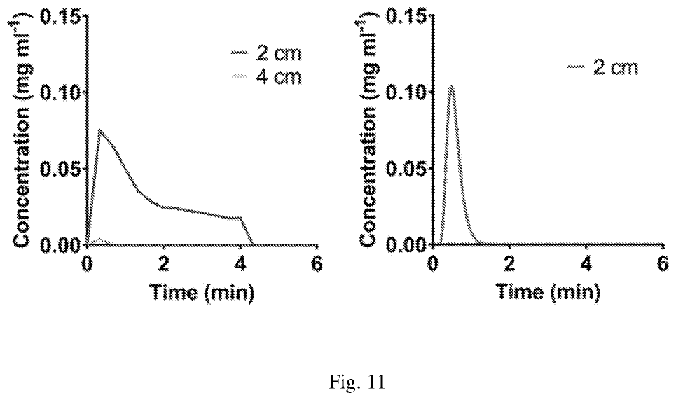

To quantify the efficiency of pesticide delivery at the root level, we solved the model for the Cy5 dispersion constant D_{P} and the rate constant of Cy5 absorption to soil k_{PS} by comparing the empirical mobility data for free Cy5 to the corresponding model output (FIG. 11). We then used the average values of D_{NP}, k_{NPS}, D_{P} and k_{PS} to optimally estimate k_{PF}, compared to the empirical data representing the nanoparticles encapsulating Cy5 (FIG. 4A-B). Again, the model output matched the empirical data closely. The rate of Cy5 release k_{PF} can be ranked from highest to lowest as follows: PhMV>>>CPMV>TMGMV>>MSNP. Interestingly, these results do not match the release profile of Cy5 in the dialysis assay (FIG. 2), suggesting that the interaction between nanoparticles and soil has a major influence on the release rate.

### Testing the Nanopesticide Model in a Real-Life Scenario

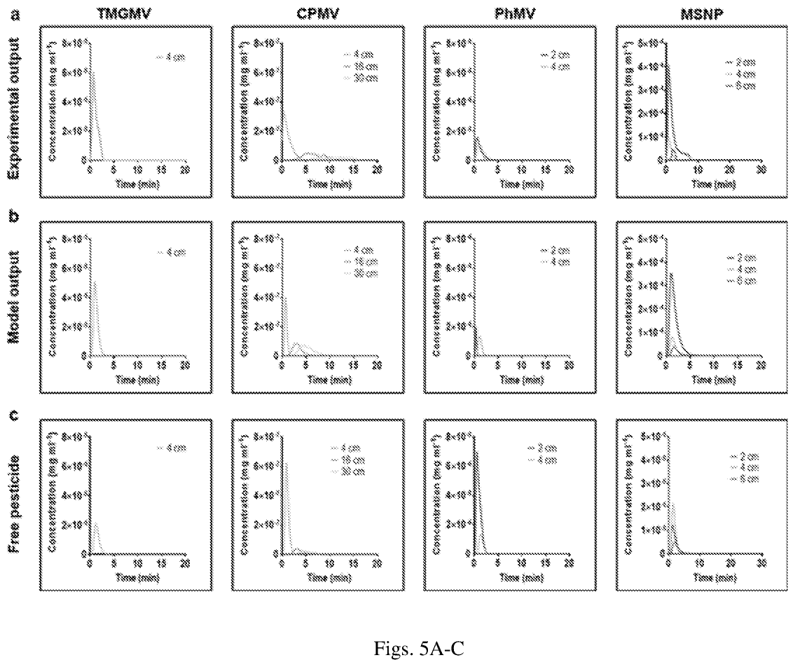

Nematode endoparasites of the genera Meloidogyne, Globodera and Heterodera infect 3,000 different plant species including many crops, and are most abundant ˜24 cm beneath the soil surface. Based on our empirical and modelling results, we selected TMGMV to deliver the nematicide abamectin, which binds to glutamate receptors in the nematode's nerve and muscle cells, causing paralysis and ultimately death. Abamectin is insoluble in water and binds strongly to organic matter in the top layer of soil, so its effect in the rhizosphere is limited and it is an ideal candidate for nanopesticide delivery using TMGMV. We used our nanopesticide model to determine how much TMGMV formulation must be applied to maintain the IC₅₀ concentration of abamectin 24 cm beneath the surface for at least 24 h. A conjugated formulation would be better than encapsulation to avoid premature release, and the linkage should be stable enough to allow the carrier to reach the target depth before the cargo is dispersed, such as a labile ester with a half-life release rate of 4 days. The IC₅₀ value of abamectin is 1.309×10⁻⁴ mg cm⁻³, and at least this concentration must therefore be achieved in the rhizosphere. We modelled flow rates of 0.1, 0.2, 0.5, 1 and 2 cm³ cm⁻² min⁻¹, representing the typical range of crop irrigation systems, and used a common irrigation regimen of 1 h three times a week, the first irrigation taking place immediately after nanopesticide application. The values of D_{NP} and k_{NPS} for TMGMV were determined as above, and in place of abamectin we used the values for the chemically similar Cy5. Because abamectin is conjugated to TMGMV, the rate constant of abamectin release from TMGMV in fluid k_{PF} was set to 0. We also assumed complete release at the root level due to the hydrolysis of the labile ester linkage over the course of a few days. The simulation output (FIG. 5) revealed that the mass of nanopesticide needed to maintain the target abamectin concentration for 24 h was dependent on the flow rate. Without no irrigation, neither free nor conjugated abamectin would achieve that concentration due to the extremely slow rate of diffusion (8.5×10⁻¹⁰ cm² min⁻¹ for TMGMV-abamectin conjugates). At a flow rate of 0.5 cm³ min⁻¹, the lowest dose of TMGMV-abamectin required to maintain the target abamectin concentration 24 cm below the surface was 0.1056 mg cm⁻². The model therefore offers a powerful tool to determine an optional dose regimen that must be use to maximize the efficacy of pesticides in the rhizosphere.

We have shown the use of VNPs/VLPs as carriers to deliver pesticides to the rhizosphere, where many pest species reside. Compared to icosahedral VNPs based on CPMV and VLPs based on PhMV, and synthetic counterparts with a similar geometry (PLGA and MSNP), the rod-like VNPs based on TMGMV achieved much greater mobility in soil and also showed the highest dye loading capacity. This is the first evidence that nanoparticles with a high aspect ratio are more mobile in the soil than spherical counterparts. In conjunction with our empirical data, we developed a computational model to predict the transport behavior of pesticides encapsulated within or conjugated to nanoparticles. This model allowed us to calculate the optimal pesticide dose that must be applied to crops in order to achieve an effective dose at root level. This precision farming approach will increase the efficacy of pesticide applications while reducing the risk of residual chemicals to human health and the environment.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A method of delivering an agricultural composition to a plant comprising:
determining a dose of the agricultural composition required to deliver a treatment effective amount of at least one agrochemical agent to a target soil depth using a computational model, the agricultural composition comprising a plurality of plant viral nanoparticles (VNPs) and/or virus-like particles (VLPs) and at least one agrochemical agent; and
applying the determined dose of the agricultural composition to the plant.

2. (canceled)

3. The method of claim 1, wherein the computational model is defined by a set of parameters comprising one or more of the following parameters, a dispersion constant of the plant viral nanoparticle (VNP) or plant virus-like particle (VLP) through soil, a dispersion constant of the agrochemical agent through soil, a rate constant of VNP or VLP absorption to soil, a rate constant of the agrochemical absorption to soil, and a rate constant of the agrochemical release from VNP or VLP in fluid.

4. The method of claim 1, wherein the treatment effective amount is the amount capable of maintaining the IC₅₀ concentration of an agrochemical agent at the target soil depth beneath the surface for at least 24 hours.

5. The method of claim 1, wherein the plant virus plant viral nanoparticles (VNPs) and/or virus-like particles (VLPs) improves the biodegradability, stability, permeability, soil mobility, and/or dispersion of the at least one agrochemical agent in soil.

6. The method of claim 1, wherein the plant VNPs are nonpathogenic or pathogenic plant virus particles.

7. The method of claim 1, wherein the plant VNPs and/or VLPs include rod-shaped plant VNPs and/or VLPs.

8. The method of claim 7, wherein the rod-shaped plant VNPs and/or VLPs include Virgaviridae virus and VLPs thereof.

9. The method of claim 7, wherein the rod-shaped plant VNPs and/or include Tobamovirus and VLPs thereof.

10. The method of claim 9, wherein the rod-shaped plant VNPs and/or VLPs include tobacco mild green mosaic virus (TMGMV) and/or VLPs thereof.

11. The method of claim 1, wherein the plant VNPs and/or VLPs include icosahedral-shaped plant VNPs and/or VLPs.

12. The method of claim 11, wherein the icosahedral-shaped plant VNPs and/or VLPs is selected from the group consisting of a plant picornavirus and Tymovirus virus.

13. The method of claim 12, wherein the picornavirus is a cowpea mosaic virus (CPMV) or VLP thereof.

14. The method of claim 12, wherein the Tymovirus virus is a physalis mottle virus (PhMV) or VLP thereof.

15. The method of claim 1 wherein the at least one agrochemical agent is conjugated to the interior and/or exterior surfaces of the plant VNPs and/or VLPs.

16. The method of claim 15, wherein the at least one agrochemical agent is covalently bound to chemically modified amino acid residues on the interior or exterior surface of the plant VNPs and/or VLPs.

17. The method of claim 16, wherein the at least one agrochemical agent is conjugated to the exterior surface of the plant VNPs and/or VLPs via a linker.

18. The method of claim 17, the linker comprising a labile ester cleavable linker.

19. The method of claim 1, wherein the at least one agrochemical is encapsulated within the interior surface of the plant VNPs and/or VLPs.

20. The method of claim 19, wherein the at least one agrochemical is encapsulated by mixing plant VNPs and/or VLPs with a molar excess of the at least one agrochemical agent.

21. The method of claim 1, wherein the target soil depth is the depth of the rhizosphere region or the root level of the plant.

22-32. (canceled)
